(19) United States
(12) Patent Application Publication
(10) Pub. No.: US 2006/0008880 A1
(43) Pub. Date: Jan. 12, 2006

(54) Mouse trace amine associated receptors and polypeptides

(76) Inventors: Martin Ebeling, Grenzach-Wyhlen (DE); Marius Hoener, Basel (CH); Lothar Lindemann, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/174,819

(22) Filed: Jul. 5, 2005

(30) Foreign Application Priority Data
Jul. 8, 2004 (EP) 04103262.4

(51) Int. Cl. C12P 21/06; C07H 21/04; C07K 14/705; C12N 5/06

(52) U.S. Cl. 435/69.1; 435/320.1; 435/354; 530/350; 536/23.5

(57) ABSTRACT

The present invention provides a fingerprint sequence which is specific and selective for trace amine associated receptors (TAAR) forming a subfamily of G protein coupled receptors. The invention also provides the novel mouse polypeptides identified as members of this family, nucleic acids encoding said polypeptides, and vectors and host cells comprising the novel family members. In addition, the invention provides methods of identifying TAARs.

### FIELD OF THE INVENTION

The present invention provides a fingerprint sequence which is specific and selective for trace amine associated receptors (TAAR) in mouse, forming a subfamily of G protein coupled receptors. The invention also provides the mouse polypeptides identified as members of this family, nucleic acids encoding said polypeptides, and vectors, host cells and non-human animals comprising the novel family members. In addition, the invention provides methods of identifying mouse TAARs.

### BACKGROUND OF THE INVENTION

Trace amines (TA) are endogenous compounds structurally related to biogenic amines and are found in the mammalian nervous system in trace amounts. TAs are stored in nerve terminals and released together with classical biogenic amines. To date there is no evidence for the existence of synapses using TAs as their exclusive transmitter. Recently, receptors specifically binding trace amines were reported by Borowski et al (Trace amines: identification of a family of mammalian G protein-coupled receptors. Proc Natl Acad Sci USA (2001) 98(16):8966-71) and Bunzow et al (Amphetamine, 3,4-methylenedioxymethamphetamine, lysergic acid diethylamide, and metabolites of the catecholamine neurotransmitters are agonists of a rat trace amine receptor. Mol Pharmacol. 2001, 60(6):1181-8.). These receptors apparently represent a novel GPCR-family.

The dysregulation of trace amines were linked to various psychiatric disorders like depression (Sandler M. et al., Decreased cerebrospinal fluid concentration of free phenylacetic acid in depressive illness. Clin Chim Acta. 1979, 93(1):169-71; Davis B A and Boulton A A. The trace amines and their acidic metabolites in depression—an overview. Prog Neuropsychopharmacol Biol Psychiatry. 1994, 18(1):17-45.), schizophrenia (Potkin S G et al., Phenylethylamine in paranoid chronic schizophrenia. Science. 1979, 206(4417):470-1; Sandler M and Reynolds G P. Does phenylethylamine cause schizophrenia? Lancet. 1976, 1(7950):70-1.) and bipolar disorder (Boulton A A.: Some aspects of basic psychopharmacology: the trace amines. Prog Neuropsychopharmacol Biol Psychiatry. 1982;6(4-6):563-70; Sabelli H C et al., Clinical studies on the phenylethylamine hypothesis of affective disorder: urine and blood phenylacetic acid and phenylalanine dietary supplements. J Clin Psychiatry. 1986 February;47(2):66-70.). There has also been made a link between dysregulation of trace amines and attention-deficit hyperactivity disorder (Baker et al., Phenylethylaminergic mechanisms in attention-deficit disorder. Biol Phychiatry. 1991, 29(1):15-22), Parkinson's disease (Heller B and Fischer E., Diminution of phenethylamine in the urine of Parkinson patients. Arzneimittelforschung. 1973, 23(6):884-6.), migraine (D'Andrea G. et al., Elusive amines and primary headaches: historical background and prospectives. Neurol Sci. 2003, 24 Suppl 2:S65-7; D'Andrea, G. et al., Elevated levels of circulating trace amines in primary headaches. Neurology. 2004, 62(10):1701-1705.) and eating disorders (Wolf M E and Mosnaim A D. Phenylethylamine in neuropsychiatric disorders. Gen Pharmacol. 1983, 14(4):385-90; Branchek T A and Blackburn T P. Trace amine receptors as targets for novel therapeutics: legend myth and tact. Curr. Opin Pharmacol. 2003. 3(1):90-97.) such as i.e. obesity and anorexia as suggested by the very high structural similarity between PEA and amphetamin, which is considered the strongest anorexic compound known to date. (Samanin R, and Garattini S.: Neurochemical mechanism of action of anorectic drugs. Pharmacol Toxicol. 1993 August;73(2):63-8; Popplewell D A et al., A behavioural and pharmacological examination of phenylethylamine-induced anorexia and hyperactivity—comparisons with amphetamine. Pharmacol Biochem Behav. 1986 Oct.;25(4):711-6.).

Therefore, there is a broad interest to increase the knowledge about trace amine receptors, especially to identify further trace amine receptors. However, examination of the literature and public database entries revealed inconsistencies in the naming of the TA receptors, e.g. the human receptor GPR102 (Lee et al., Discovery and mapping of ten novel G protein-coupled receptor genes. Gene. 2001, 275(1):83-91.) is also referred to as TA5 (Borowski et al Trace amines: identification of a family of mammalian G protein-coupled receptors. Proc Natl Acad Sci USA (2001) 98(16):8966-71)), and human 5-HT4Ψ (Liu et al., A serotonin-4 receptor-like pseudogene in humans. Brain Res Mol Brain Res. 1998, 53(1-2):98-103.) has also been named TA2Ψ (Borowski et al., Trace amines: identification of a family of mammalian G protein-coupled receptors. Proc Natl Acad Sci USA (2001) 98(16):8966-71). In addition, GPR57 (Lee et al., Cloning and characterization of additional members of the G protein-coupled receptor family. Biochim Biophys Acta. 2000, 1490(3):311-23.), GPR58 (Lee et al., Cloning and characterization of additional members of the G protein-coupled receptor family. Biochim Biophys Acta. 2000, 1490(3):311-23.) and PNR (Zeng et al., Cloning of a putative human neurotransmitter receptor expressed in skeletal muscle and brain. Biochem Biophys Res Commun. 1998, 242(3):575-8.) were so far not generally recognized as TA receptor. This inconsistencies cause confusion and uncertainty. Therefore, there is a strong need for a clear definition of this receptor family.

### SUMMARY OF THE INVENTION

The present invention provides a fingerprint sequence which is specific and selective for trace amine associated receptors (TAAR) in mouse, forming a subfamily of G protein coupled receptors. The invention also provides the mouse polypeptides identified as members of this family, nucleic acids encoding said polypeptides, and vectors, host cells and non-human animals comprising the novel family members. In addition, the invention provides methods of identifying mouse TAARs.

In order to resolve any ambiguity of the current naming a novel, uniform nomenclature is proposed with this invention which refers to these receptors as Trace Amine Associated Receptors (TAARs). This novel nomenclature reflects the finding that at least some TAARs do not respond to TAs at all, hence the term “associated”. It covers all receptors of this GPCR family and is compatible with the different number of receptor genes in different species. The novel nomenclature is strictly based on the sequential order of the receptor genes on the respective human chromosome as well as a detailed phylogenetic analysis (FIG. 3) of the receptor genes across different species. The nomenclature adheres to the following rules: Any two (or three) genes that are orthologues, i.e. that were generated through a speciation event, should be labeled with the same number. Vice versa, two genes must not have the same number if they are not orthologous.

Genes that are paralogues, i.e. that were generated through a gene duplication event within the lineage of one species, should be distinguished by a letter suffix.

Examples: Genes hTAAR 5, rTAAR 5, mTAAR 5 are all orthologues.
Genes mTAAR 8a,b,c in mouse are paralogues.
Genes mTAAR 8a,b,c are all orthologues to hTAAR 8.

The present invention provides novel polypeptides identified as TAAR family members, the use of said polypeptides as drug target, the polynucleotide sequences encoding said polypeptide, and vectors, host cells and non-human animals comprising said polynucleotides. Furthermore, the present invention pertains the fingerprint motif specific and selective for the TAAR family and the use of it.

### DESCRIPTION OF THE FIGURES

FIG. 1 shows the chromosomal localization of TAARs genes in mouse. The relative position of TAAR genes on the chromosomes is indicated by boxes, representing the single coding exons of the respective genes. For mTAAR 2, which is encoded by two exons, the box indicates the position of the second coding exon harboring more than 95% of the open reading frame. The width of the boxes is not to scale with regard to the length of the respective coding sequences, and the colors indicate a distinction in receptor subgroups as discussed in the text. Arrows on top of the boxes indicate the orientation of the mTAAR genes (→: ORF in direction of the forward strand, ←: ORF in direction of the reverse strand). (Ψ=Pseudogene)

FIG. 2 shows an alignment of all functional TAARs. Amino acid residues conserved in all human, rat and mouse TAARs are highlighted by black shading. The characteristic TAAR fingerprint motif is located in TM VII.

A: alignment of functional human TAARs. Predicted TM Positions: hTAAR 1 (TM I: 26-46, TM II: 60-80, TM III: 99-109, TM IV: 137-157, TM V: 189-209, TM VI: 253-273, TM VII: 288-308); hTAAR 2 (TM I: 49-71, TM II: 80-102, TM III: 129-151, TM IV: 163-185, TM V: 208-230, TM VI: 263-285, TM VII: 300-322); hTAAR 5 (TM I: 36-58, TM II: 71-93, TM III: 108-130, TM IV: 150-172, TM V: 203-225, TM VI: 253-270, TM VII: 285-307); hTAAR 6 (TM I: 33-53, TM II: 69-89, TM III: 108-128, TM IV: 148-168, TM V: 203-223, TM VI: 260-276, TM VII: 283-302); hTAAR 8 (TM I: 37-59, TM II: 66-88, TM III: 103-125, TM IV: 145-167, TM V: 198-220, TM VI: 258-280, TM VII: 290-312); hTAAR 9 (TM I: 38-58, TM II: 69-89, TM III: 107-128, TM IV: 148-168, TM V: 198-218, TM VI: 260-280, TM VII: 295-315)

B: alignment of functional mouse TAARs.

C: alignment of functional rat TAARs.

FIG. 3 shows the phylogenetic relationship of TAAR genes in human, rat and mouse based on their DNA sequence. Subsequent gene duplication events are indicated by numbers at the branching points ( [symbol] : Gene duplication from a putative common ancestor; [symbol] : Speciation leading to separate primates and rodent lineages; [symbol] : Gene duplication leading to mouse and rat species). The outgroup was placed based on sequence comparison to the human 5-HT₄ receptor. The summary of TAAR genes into distinct subfamilies is indicated by boxes with grey shading. Ψ: Pseudogene; *: Gene fragment with closest similarity to rat TAAR 7h in the human genome resembling remnants of rodent TAAR 7 paralogues. Scale: JTT protein distances.

FIG. 4 shows the “repair” of the pseudogene hTAAR 3 (old name GPR57ψ): The two bps CC in position 133-134 of the fixed ORF were added, which repairs the otherwise early stop codon TCA.

FIG. 5 shows the relationship of TAARs based on the properties of the ligand pocket vectors (amino acid whose residues probably take part in the ligand binding, determined by calculation model).

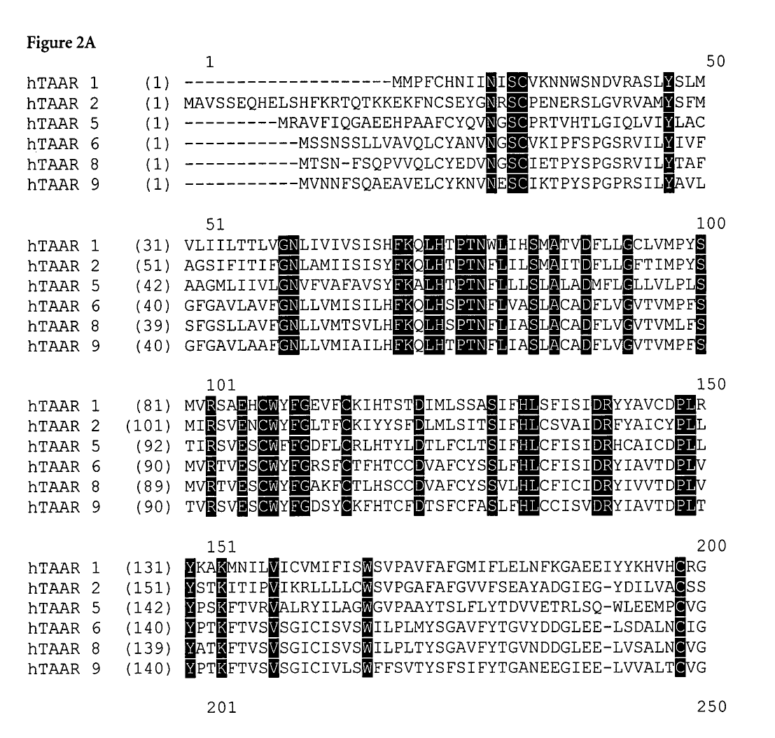

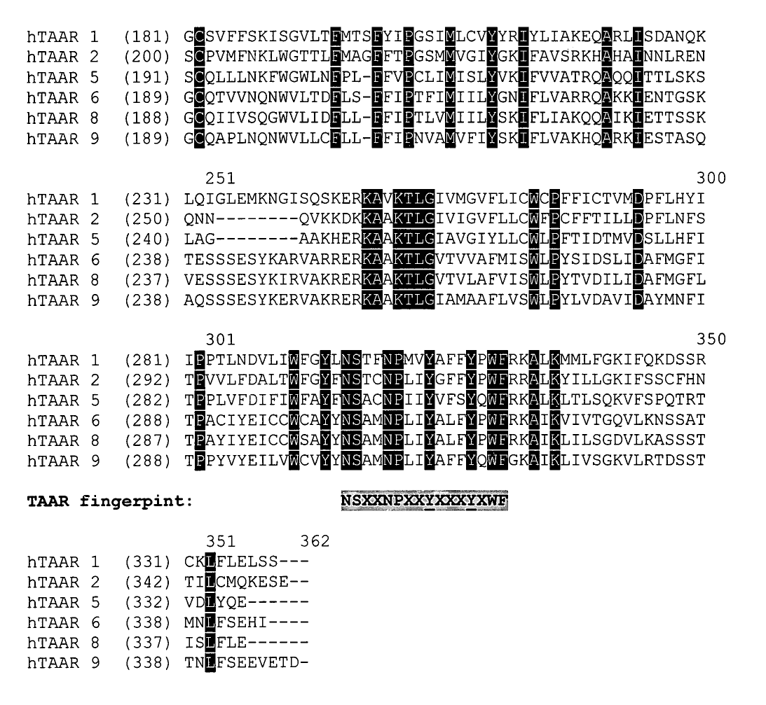

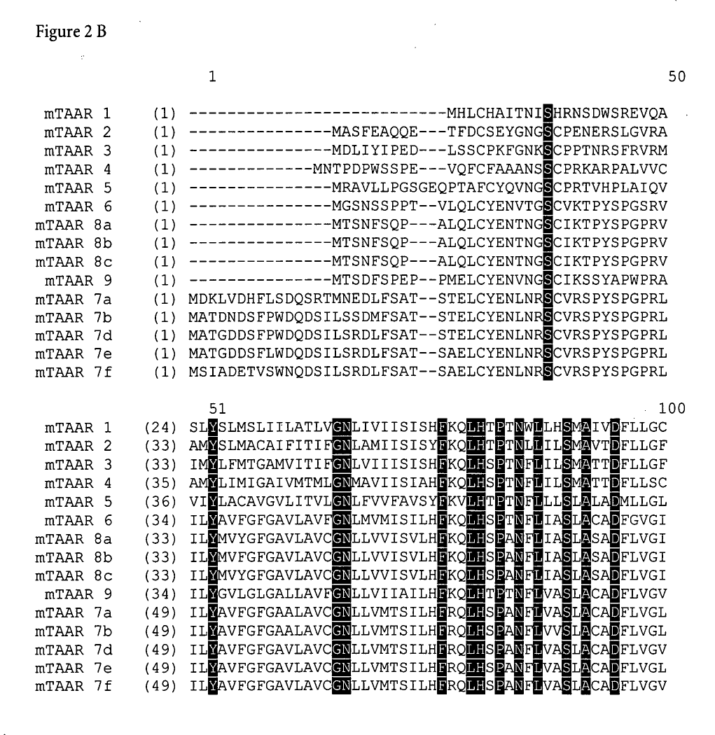

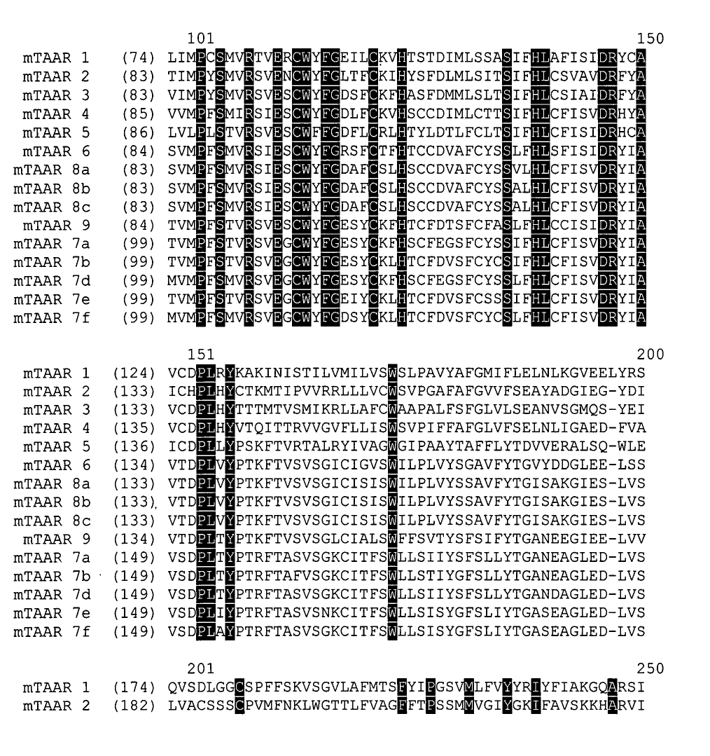

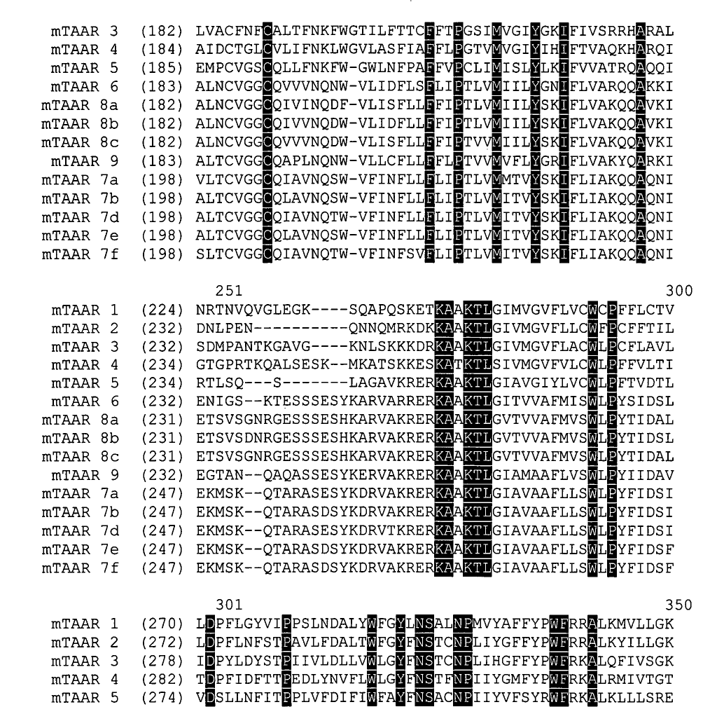

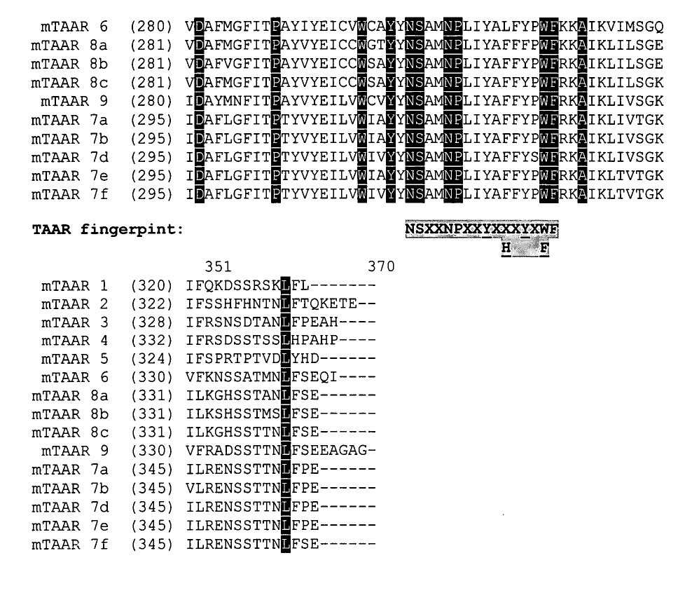

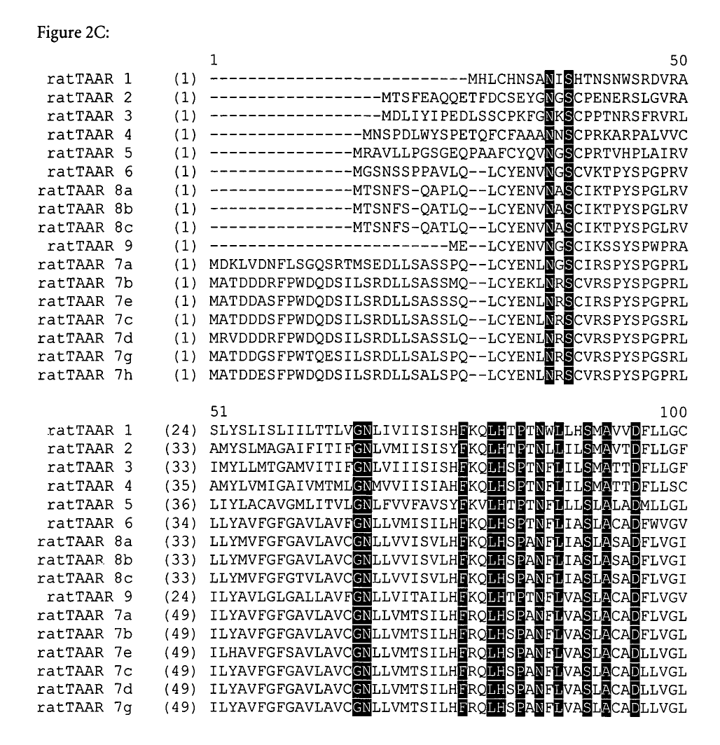

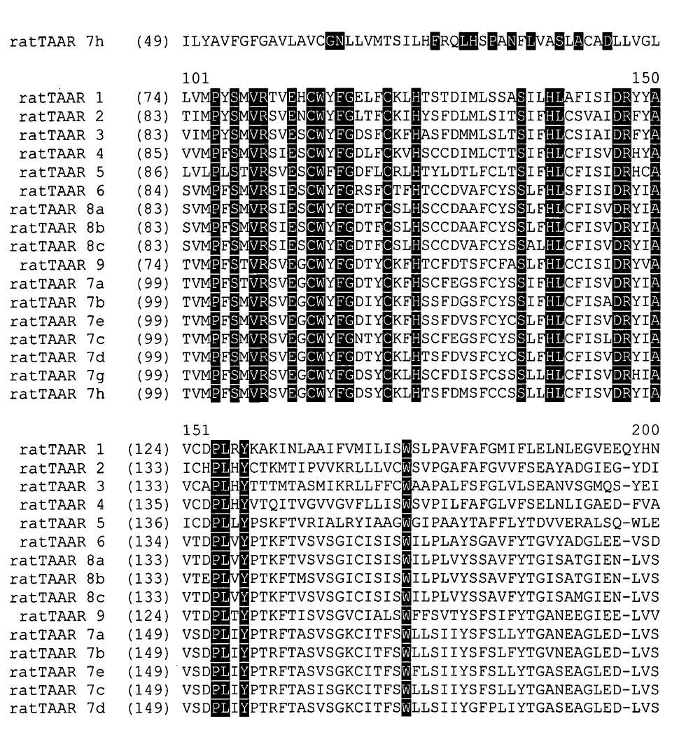

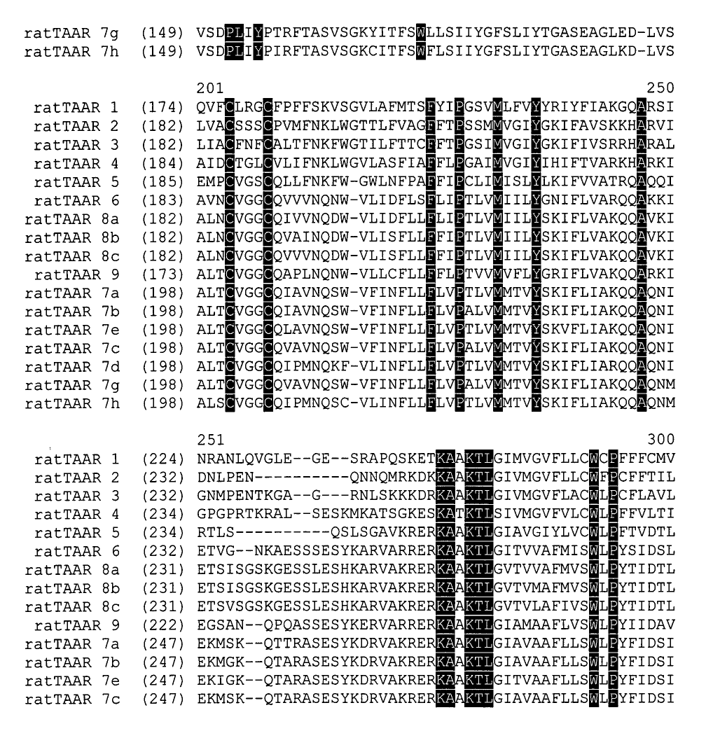

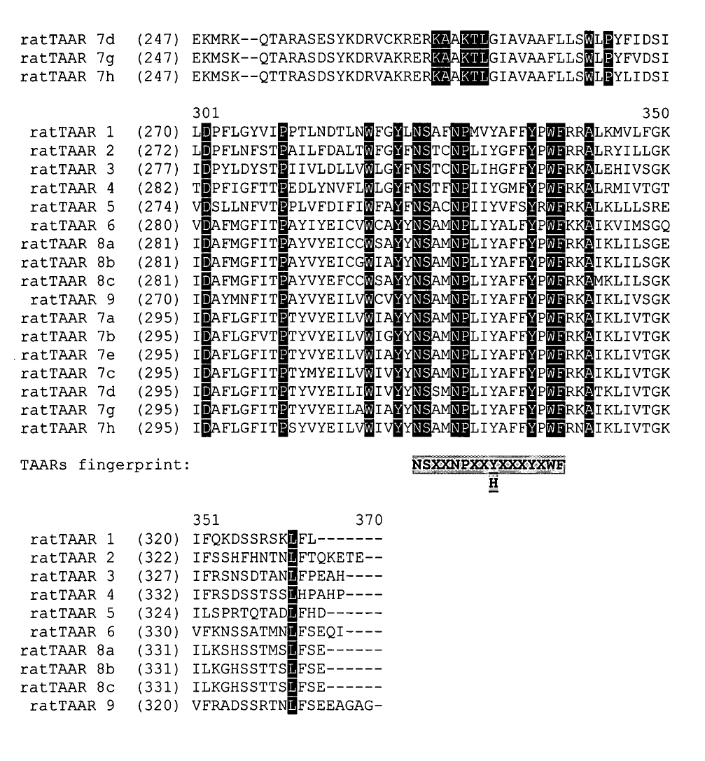

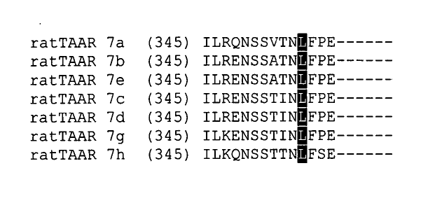

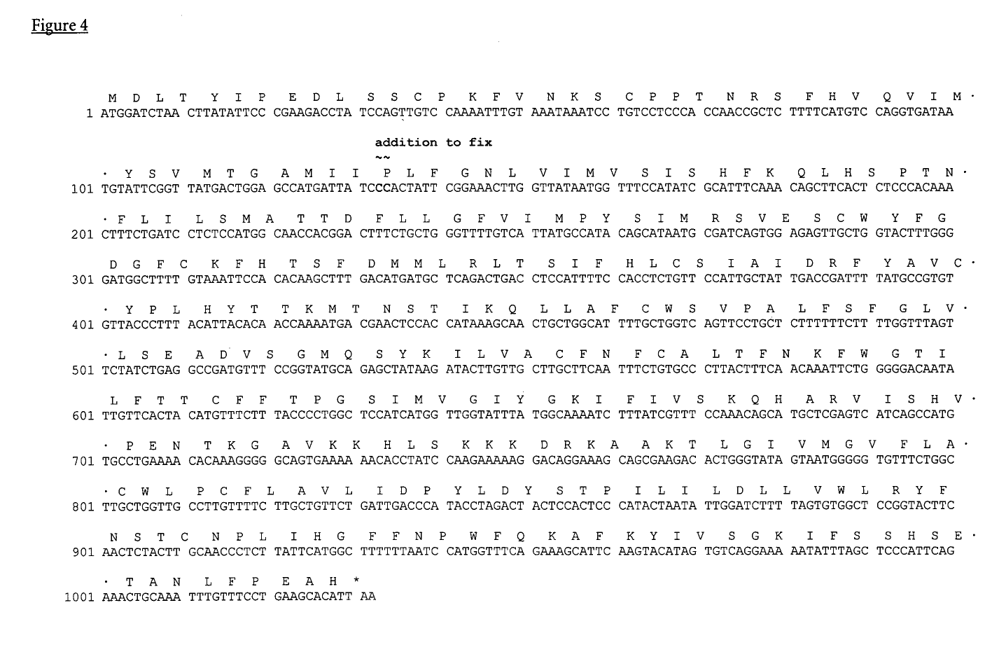

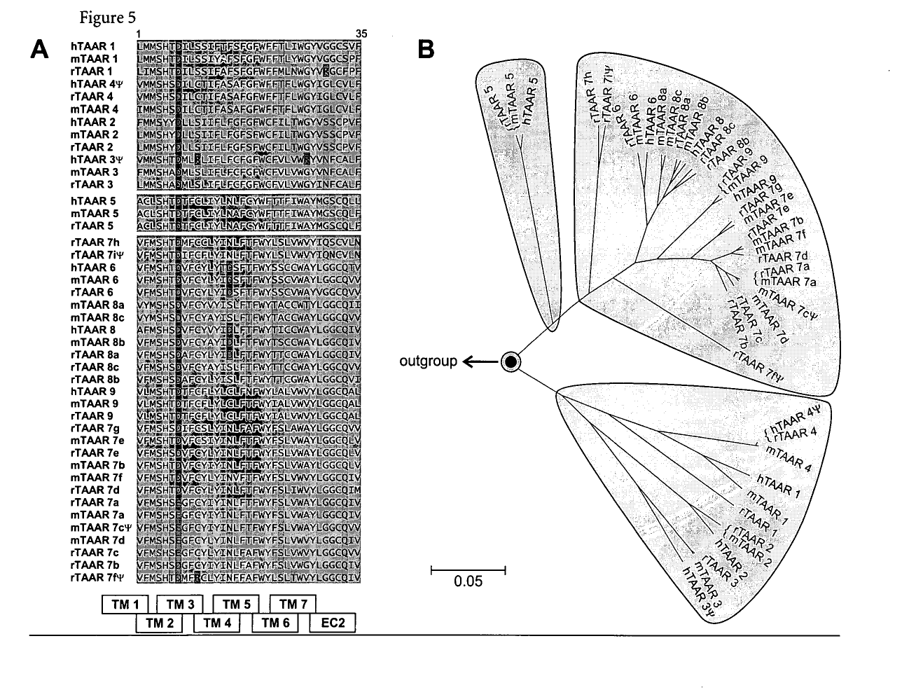

A: The Ligand Pocket Vectors (LPVs) of human, rat and mouse TAAR proteins, as predicted according to Kratochwil et al. (2004). Individual TAARs are ordered such that proteins with closest similar ligand binding pockets are placed next to each other. The physicochemical properties of the amino acid residues are indicated by colored shading (blue: hydrophobic/aromatic residues; green: aliphatic polar residues; red/plum: positively/negatively charged residues; orange/yellow: glycine/proline residues, potentially inducing kinks in the TM domains; pink: histidine; light blue: tyrosine). The position of the respective amino acids in the transmembrane domains (TM 1-7) as well as the extracellular loop II (EC II) is indicated by boxes in the lower part of the figure. For the precise position of predicted TM regions please refer to legend of FIG. 2.

B: Hierarchical tree representation of the pharmacophore similarity of the ligand binding pockets of TAAR proteins. The following proteins were included into the analysis as “repaired pseudogenes”, modification in parenthesis: hTAAR 3Ψ (insertions: C135-, A136-), hTAAR 4Ψ (pointmutation: T411A, deletions: -605G, -750A), mTAAR 7cΨ (insertion: C513-), rTAAR 7fΨ (pointmutation: G515A), rTAAR 7iΨ (insertion: A90-). Scale bar: Pharmacophore diversity units.

### Definitions

As used herein, the singular form of any term also encompasses the plural form and vice versa.

The term “gene” as used herein refers to any segment of DNA associated with a biological function. A gene is an ordered sequence of nucleotides located in a particular position on a particular chromosome that encodes a specific functional product.

The term “pseudogene” as used herein relates to an inactive gene which may have evolved by mutation events from an active ancestor gene. Inactive means that the gene is not translated to functional polypeptide.

The term “polypeptide sequence” (e.g., a protein, polypeptide, peptide, etc.) as used herein relates to a polymer of amino acids comprising naturally occurring amino acids.

The term “isolated” means altered “by the hand of man” form the natural state. If an “isolated” composition or substance occures in nature, it has been changed or removed from its orignial environment, or both. For example, a polynucleotide or a polypeptide naturally present in a living animal is not “isolated”, but the same polynucleotide or polypeptide separated form the coexisting materials of its natural state is “isolated” as the term is employed herein.

The term “recombinant” when used with reference, e.g. to a polynucleotide or polypeptide typically indicates that the polynucleotide or polypeptide has been modified by the introduction of a heterologuous (or foreign) nucleic acid of the alteration of a native nucleic acid, or that the protein or polypeptide has been mdified by the introduction of a hteerologous amino acid.

The term “polynucleotide sequence” (e.g., a nucleic acid, polynucleotide, oligonucleotide, etc.) as used herein relates to a polymer of nucleotides comprising nucleotides A,C,T,U,G.

The term “polypeptide of the invention” relates to the novel polypetides provided in the present invention, i.e. mTAAR2 to mTAAR9.

### DETAILED DESCRIPTION OF THE INVENTION

The present invention provides novel polypeptides identified as TAAR family members, the use of said polypeptides as drug target, the polynucleotide sequences encoding said polypeptide, and vectors, host cells and non-human animals comprising said polynucleotides. Furthermore, the present invention pertains the fingerprint motif specific and selective for the TAAR family and the use of it.

The present invention provides fingerprint motif comprising the sequence NSXXNPXXZXXXBXWF (SEQ. ID NO: 1), wherein X is any natural occurring amino acid and Z may be a tyrosine or histidine and B may be a tyrosine or phenylalanine and wherein the amino acid abbreviations N,S, P, W and F are known to one of ordinary skill in the art as representing asparagine, serine, proline, tryptophan and phenylalanine respectively. The term “fingerprint”, “fingerprint motif” or “fingerprint sequence” as used herein relates to an amino acid sequence which is specific and selective for the mouse GPCR subfamily TAAR. Preferably, the fingerprint motif is specific for functional TAARs. The tryptophan residue in the context of the fingerprint motif is found exclusively in TAARs and not in any other known GPCR; rather, the corresponding sequence position is almost invariably occupied by polar or even charged amino acids in other GPCRs. The fingerprint motif may be used for identifying TAARs, preferably for identifying functional TAARs.

The present invention also provides a method of identifying mouse TAARs using the fingerprint sequence (SEQ. ID NO: 1). To be identified as a member of the mouse TAARs family a polypeptide may have 100% identity.

Furthermore, the present invention pertains a method of identifying TAARs of other species, preferably of mammalians, using the fingerprint sequence (SEQ. ID NO: 1). To be identified as a member of the mammalian TAARs family a polypeptide may have at least 75% identity with the fingerprint sequence, preferably more than 87% identity, more preferably 100% identity.

The fingerprint sequence may be used as a “query sequence” to perform a search against sequence databases to, for example to identify TAAR family members. Such searches can be performed using a pattern recognition program as for example fuzzpro of EMBOSS (Rice et al., EMBOSS: the European Molecular Biology Open Software Suite. Trends in Genetics, 2000, 16(6):276-277). A search for mouse TAAR may be performed with fuzzpro, NSXXNPXX[HY]XXX[YF]XWF as the required sequence, number of mismatches=0, database=swissprot (release 43).

The mouse TAAR family contains sixteen members (mTAAR1 to mTAAR 9) wherein one of them is a pseudogene: mTAAR7cΨ. All genes encoding the mouse TAAR family members are located in the same region of chromosome 10 (10A4). With the exception of mTAAR 2, which is encoded by two exons, the coding sequences of all TAAR genes are located in a single exon.

The present invention provides an isolated or recombinant polypeptide comprising sequence SEQ. ID NO: 5. Said polypeptide was identified as trace amine associated receptor (TAAR) and was named mTAAR2.

The present invention further pertains the polynucleotide sequence comprising SEQ. ID NO: 4 that encodes the polypeptide mTAAR2.

The present invention provides further an isolated or recombinant polypeptide comprising sequence SEQ. ID NO: 7. Said polypeptide was identified as trace amine associated receptor and was named mTAAR3.

The present invention also pertains the polynucleotide sequence comprising SEQ. ID NO: 6 that encodes the polypeptide mTAAR3.

The present invention provides further an isolated or recombinant polypeptide comprising sequence SEQ. ID NO: 9. Said polypeptide was identified as trace amine associated receptor and was named mTAAR4.

The present invention also pertains the polynucleotide sequence comprising SEQ. ID NO: 10 that encodes the polypeptide mTAAR4.

The present invention provides further an isolated or recombinant polypeptide comprising sequence SEQ. ID NO: 11. Said polypeptide was identified as trace amine associated receptor and was named mTAAR5.

The present invention also pertains the polynucleotide sequence comprising SEQ. ID NO: 10 that encodes the polypeptide mTAAR5.

The present invention provides further an isolated or recombinant polypeptide comprising sequence SEQ. ID NO: 13. Said polypeptide was identified as trace amine associated receptor and was named mTAAR6.

The present invention also pertains the polynucleotide sequence comprising SEQ. ID NO: 12 that encodes the polypeptide mTAAR6.

The present invention provides further an isolated or recombinant polypeptide comprising sequence SEQ. ID NO: 15. Said polypeptide was identified as trace amine associated receptor and was named mTAAR7a.

The present invention also pertains the polynucleotide sequence comprising SEQ. ID NO: 14 that encodes the polypeptide mTAAR7a.

The present invention provides further an isolated or recombinant polypeptide comprising sequence SEQ. ID NO: 17. Said polypeptide was identified as trace amine associated receptor and was named mTAAR7b.

The present invention also pertains the polynucleotide sequence comprising SEQ. ID NO: 16 that encodes the polypeptide mTAAR7b.

The present invention provides further an isolated or recombinant polypeptide comprising sequence SEQ. ID NO: 22. Said polypeptide was identified as trace amine associated receptor and was named mTAAR7d.

The present invention also pertains an isolated or recombinant polynucleotide sequence comprising SEQ. ID NO: 21 that encodes the polypeptide mTAAR7d.

The present invention provides further an isolated or recombinant polypeptide comprising sequence SEQ. ID NO: 24. Said polypeptide was identified as trace amine associated receptor and was named mTAAR7e.

The present invention also pertains an isolated or recombinant polynucleotide sequence comprising SEQ. ID NO: 23 that encodes the polypeptide mTAAR7e.

The present invention provides further an isolated or recombinant polypeptide comprising sequence SEQ. ID NO: 26. Said polypeptide was identified as trace amine associated receptor and was named mTAAR7f

The present invention also pertains an isolated or recombinant polynucleotide sequence comprising SEQ. ID NO: 25 that encodes the polypeptide mTAAR7f.

The present invention provides further an isolated or recombinant polypeptide comprising sequence SEQ. ID NO: 28. Said polypeptide was identified as trace amine associated receptor and was named mTAAR8a.

The present invention also pertains an isolated or recombinant polynucleotide sequence comprising SEQ. ID NO: 27 that encodes the polypeptide mTAAR8a.

The present invention provides further an isolated or recombinant polypeptide comprising sequence SEQ. ID NO: 30. Said polypeptide was identified as trace amine associated receptor and was named mTAAR8b.

The present invention also pertains an isolated or recombinant polynucleotide sequence comprising SEQ. ID NO: 29 that encodes the polypeptide mTAAR8b.

The present invention provides further an isolated or recombinant polypeptide comprising sequence SEQ. ID NO: 32. Said polypeptide was identified as trace amine associated receptor and was named mTAAR8c.

The present invention also pertains an isolated or recombinant polynucleotide sequence comprising SEQ. ID NO: 31 that encodes the polypeptide mTAAR8c.

The present invention provides further an isolated or recombinant polypeptide comprising sequence SEQ. ID NO: 34. Said polypeptide was identified as trace amine associated receptor and was named mTAAR9.

The present invention also pertains an isolated or recombinant polynucleotide sequence comprising SEQ. ID NO: 33 that encodes the polypeptide mTAAR9.

The other member of the TAARs family mTAAR1 having the polynucleotide sequence SEQ. ID NO: 2 and the polypeptide sequence SEQ. ID NO: 3, was previously described (Borowsky, B et al., Trace amines: identification of a family of mammalian G protein-coupled receptors, Proc. Natl. Acad. Sci. U.S.A. (2001)98 (16), 8966-8971).

The present invention also provides the nucleotide sequences of mTAAR7cΨ (SEQ. ID NO: 18). Furthermore, the present invention provides the repaired nucleotide sequences of mTAAR7cΨ (SEQ. ID NO: 19). This invention further provides the polynucleotide sequences encoded by the fixed nucleotide sequence mTAAR7c (SEQ. ID NO: 20). The term “repaired gene” or “fixed gene” as used herein relates to a pseudogene whose sequence was altered in a way that the gene becomes active. The polynucleotide sequence of i.e. hTAAR3 is inactive due to a mutation that caused an early stop codon. The sequence was repaired by insertion of two nucleotides at position 133-134 (see FIG. 4).

The present invention also provides isolated or recombinant polypeptides comprising an amino acid sequence which comprise the fingerprint sequence (SEQ. ID NO: 1) and differs from the sequence SEQ. ID NO: 3.

In a further aspect the invention relates to the use of the polypeptides of the invention as drug target. Preferably, the polypeptides of the invention are used as a drug target for identifying compounds useful in depression therapy, in schizophrenia therapy, in migraine therapy, or in therapy of attention-deficit hyperactivity disorder or eating disorder as anorexia or obesity. The drug target may be suitable for the design, screening and development of pharmaceutically active compounds.

One embodiment of the invention relates to the polypeptide having a polypeptide sequence SEQ. ID NO: 5, 7, 9, 11, 13, 15, 17, 22, 24, 26, 28, 30, 32 or 34 as drug target. Preferably, the polypeptides of the invention are used as a drug target for identifying compounds useful in depression therapy, in schizophrenia therapy, in migraine therapy, or in therapy of attention-deficit hyperactivity disorder or eating disorder as anorexia or obesity.

Another embodiment of the invention relates to mouse receptors having an amino acid sequence which comprise the fingerprint sequence and differs from the sequences SEQ. ID NOs: 3 as drug target. Preferably, these receptors are drug target for identifying compounds useful in depression therapy, in schizophrenia therapy, in migraine therapy, or in therapy of attention-deficit hyperactivity disorder or eating disorder as anorexia or obesity.

The present invention also pertain vectors comprising polynucleotides of the invention, host cells which are transduced with said vectors and the production of the polypeptides of the invention by recombinant techniques. Preferably, the vector comprises the polynucleotide comprising one or more of (or selected from the group consisting of) SEQ. ID NO: 4, SEQ. ID NO: 6, SEQ. ID NO: 8, SEQ. ID NO: 10, SEQ. ID NO: 12, SEQ. ID NO: 14, SEQ. ID NO: 16, SEQ. ID NO: 19, SEQ. ID NO: 21, SEQ. ID NO: 23, SEQ. ID NO: 25, SEQ. ID NO: 27, SEQ. ID NO: 29, SEQ. ID NO: 31 or SEQ. ID NO: 33 and preferably, the host cells are transduced with said vector.

Host cells can be genetically engineered (i.e., transduced, transformed or transfected) to incorporate expression systems or portion thereof for polynucleotides of the present invention. Introduction of the vector into host cells can be effected by methods described in many standard laboratory manuals, such as Davis et al., Basic methods in molecular biology Elsevier, N.Y. (1986); Davis J M (ed.): Basic cell culture: a practical approach, sec. edition. Oxford University Press (2002); R. Ian Freshney: Culture of Animal Cells: A Manual of Basic Technique, fourth edition. John Wiley & Sons (Sd) 1999; and Sambrook et al., Molecular cloning: a laboratory manual, 2. Ed., Cold Spring Harbour Laboratory Press, Cold Spring Harbor, N.Y (1989), such as calcium phosphate transfection, DEAE-dextran mediated transfection, transvectin, microinjection, cationic lopid-mediated transfection, electroporation, transduction or infection.

A host cell may be mammalian cell as HEK 293, CHO, COS, HeLa, neuronal, neuroendicrinal, neuroblastomal or glial cell lines like SH-SY5Y, PC12, HN-10 (Lee H J, Hammond D N, Large T H, Roback J D, Sim J A, Brown D A, Otten U H, Wainer B H.: Neuronal properties and trophic activities of immortalized hippocampal cells from embryonic and young adult mice. J Neurosci. 1990 June;10(6):1779-87.), IMR-32, NB41A3, Neuro-2a, TE 671, primary neuronal or glia cells from mammals like rat or mouse, Xenopus oocytes, bacterial cells such as streptococci, staphylocooci, E. coli, Streptomyces and Bacillus subtilis cells; fungal cells such as Saccharomyces cerevisiae and Aspergillus cell; insect cells such as Drosophila S2 and Spodoptera Sf9 cells and plant cells.

A great variety of expression systems can be used. Such a system include, among others, chromosomal, episomal and virus-derived systems, i.e. vectors derived from bacterial plasmids, from bacteriophage, from transposons, from yeast episoms, from yeast chromosomal elements, from viruses such as baculovirus, papova viruses, such as SV40, vaccinia viruses, adenovirus, fowl pox virus, pseudorabies, retroviruses and vectors derived from combinations thereof, such as those derived from plasmids and bacteriophage genetic elements, such as cosmids and phagemids. The expression systems may contain control regions that regulate as well as engender expression. Generally, any system or vector suitable to maintain, progagate or express polynucleotides to produce a polypeptide in a host may be used. The appropriate nucleotide sequence may be inserted into an expression system by any of a variety of well-known and routine techniques, such as, for example, those set forth on Sambrook et al., Molecular cloning: a laboratory manual, 2. Ed., Cold Spring Harbour Laboratory Press, Cold Spring Harbour, N.Y. (1989) or Borowski et al Trace amines: identification of a family of mammalian G protein-coupled receptors. Proc Natl Acad Sci USA (2001) 98(16):8966-71), both of which are hereby incorporated by reference.

The present invention further provides a transgenic non-human animal comprising a polynucleotide encoding a polypeptide of the invention. Preferably, the transgenic non-human animal comprises a polynucleotide comprising one or more of (or selected from the group consisting of) SEQ. ID NO: 4, SEQ. ID NO: 6, SEQ. ID NO: 8, SEQ. ID NO: 10, SEQ. ID NO: 12, SEQ. ID NO: 14, SEQ. ID NO: 16, SEQ. ID NO: 19, SEQ. ID NO: 21, SEQ. ID NO: 23, SEQ. ID NO: 25, SEQ. ID NO: 27, SEQ. ID NO: 29, SEQ. ID NO: 31 or SEQ. ID NO: 33.

The transgenic non-human animal may be any non-human animal known in the art. Preferably, the transgenic non-human animal is a mammal, more preferably the transgenic non-human animal is a rodent. Most preferably, the transgenic animal of the invention is a mouse.

Methods of producing a transgenic non-human animal are well known in the art such as for example those set forth on Hogan, B. C., F; Lacy, E, Manipulating the Mouse Embryo: A Laboratory Manual. 1986, New York: Cold Spring Harbor Laboratory Press; Hogan, B., et al., Manipulating the mouse embryo. 1994, Second Edition, Cold Spring Harbor Press, Cold Spring Harbor and Joyner, A. (ed.): Gene Targeting—A Practical Approach Second Edition. Practical Approach Series, Oxford University Press 1999, all of which are hereby incorporated by reference.

The polypeptides of the invention may be employed in a screening process for compounds (candidate compounds) which bind the receptor and which activate (agonists) or inhibit (antagonists) activation of the receptor polypeptide of the present invention or compounds which modulate receptor function e.g by acting on receptor trafficking. Candidate compounds are any compound of interest that one skilled in the art may wish to test to identify whether said compound would bind (to) the receptor and/or would activate (agonist compounds) or inhibit (antagonist compounds) activation of the receptor polypeptide at the invention (polypeptide D).

The present invention provides a method for identifying compounds which bind to a polypeptide of the invention comprising:
a) contacting the polypeptide of the invention with a candidate compound and
b) determining whether said compound binds to the polypeptide of the invention.

The invention also provides a method for identifying compounds which modulate, or in other words have a stimulatory or inhibitory effect on, the biological activity of a polypeptide of the invention or its expression comprising
a) contacting the polypeptide of the invention with a candidate compound and
b) determining if said compound has modulated the function or activity of the polypeptide of the invention.

Thus, the invention provides a method for identifying compounds which bind to a polypeptide of the invention comprising:
a) contacting the polypeptide, wherein the polypeptide D selected from the group consisting of SEQ ID NOs 5, 7, 9, 11, 13, 15, 17, 22, 24, 26, 28, 30, 32, 34 or the sequence NSXXNPXXZXXXBXWF, wherein X is any natural occurring amino acid, Z is selected from the group consisting of tyrosine or histidine and B is selected from the group consisting of tyrosine or phenylalanine, with a candidate compound and
b) determining whether said candidate compound binds to said polypeptide.

The invention also thus provides a method for identifying compounds which modulate or have a stimulatory or inhibitory effect on the biological activity of a polypeptide of the invention comprising
a) contacting a polypeptide wherein the polypeptide D selected from the group consisting of SEQ ID NOs 5, 7, 9, 11, 13, 15, 17, 22, 24, 26, 28, 30, 32, 34 or the sequence NSXXNPXXZXXXBXWF, wherein X is any natural occurring amino acid, Z is selected from the group consisting of tyrosine or histidine and B is selected from the group consisting of tyrosine or phenylalanine, with a candidate compound and
b) determining if said candidate compound has modulated the function or activity of said polypeptide.

In general, such screening procedures as described above involve producing appropriate cells which express the receptor polypeptide of the present invention on the surface thereof, however, the receptor expressed in appropiate cells may also be localized intracellularly. Such cells include i.e. cells from mammals, Xenopus, yeast, Drosophila or E. coli. Cells expressing the receptor (or cell membrane containing the expressed receptor) are then contacted with a test (candidate) compound to measure binding, or stimulation or inhibition of a functional response.

One screening technique includes the use of cells which express receptor of this invention (for example, transfected CHO cells or HEK293) in a system which measures extracellular pH or intracellular calcium changes caused by receptor activation. In this technique, candidate (potential) compounds may be contacted with cells expressing the receptor polypeptide of the present invention. A second messenger response, e.g., signal transduction, pH changes, PI hydrolyse, GTP-γ-[³⁵] release or changes in calcium level, is then measured to determine whether the potential compound activates or inhibits the receptor.

Another method involves screening for receptor inhibitors by determining inhibition or stimulation of receptor-mediated cAMP and/or adenylate cyclase accumulation. Such a method involves transfecting a eukaryotic cell with the receptor of this invention to express the receptor on the cell surface. The cell is then exposed to potential antagonists in the presence of the receptor of this invention. The amount of cAMP accumulation is then measured. If the potential antagonist binds the receptor, and thus inhibits receptor binding, the levels of receptor-mediated cAMP, or adenylate cyclase, activity will be reduced or increased.

Another method for detecting agonists or antagonists for the receptor of the present invention is the yeast based technology as described in U.S. Pat. No. 5,482,835, which is hereby incorporated by reference.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed. Standard methods for conducting such screening assays are well understood in the art.

Examples of potential polypeptide of the invention antagonists include antibodies or, in some cases, oligonucleotides or proteins which are closely related to the ligand of the polypeptide of the invention, e.g., a fragment of the ligand, or small molecular weight molecules such as e.g. metabolites of neurotransmitters or of amino acids, which bind to the receptor but do not elicit a response, so that the activity of the receptor is prevented.

Having now generally described this invention, the same will become better understood by reference to the specific examples, which are included herein for purpose of illustration only and are not intended to be limiting unless otherwise specified, in connection with the following figures.

### EXAMPLES

Commercially available reagents referred to in the examples were used according to manufacturer's instructions unless otherwise indicated.

### Example 1

### Identification of TAAR Family Members

The TAAR genes were identified by comparison of the previously published TAAR receptor genes with the human, rat and mouse genomic sequence information available from Genbank . . . see also Table 1) employing standard algorithms like BLAST.

TABLE 1
The new nomenclature for Trace Amine
Associated Receptors (TAARs).

| | | | | | SEQ. | |
|---|---|---|---|---|---|---|
| New | Old | | | Discrep- | ID NO: | |
| Name | Name | bp | Acc. No | ancy | nt. | aa. |
| mTAAR 1 | TA1 | 999 | AF380187 | — | 2 | 3 |
| mTAAR 2 | Novel | 1020 | — | — | 4 | 5 |
| mTAAR 3 | Novel | 1032 | — | — | 6 | 7 |
| mTAAR 4 | Novel | 1044 | — | — | 8 | 9 |
| mTAAR 5 | Novel | 1014 | — | — | 10 | 11 |
| mTAAR 6 | Novel | 1038 | — | — | 12 | 13 |
| mTAAR 7a | Novel | 1077 | — | — | 14 | 15 |
| mTAAR 7b | Novel | 1077 | — | — | 16 | 17 |
| mTAAR 7c ψ | Novel ψ | 1055 | — | — | 18 | — |
| mTAAR 7d | Novel | 1077 | — | — | 21 | 22 |
| mTAAR 7e | Novel | 1077 | — | — | 23 | 24 |
| mTAAR 7f | Novel | 1077 | | — | 25 | 26 |
| mTAAR 8a | Novel | 1035 | — | — | 27 | 28 |
| mTAAR 8b | Novel | 1035 | — | — | 29 | 30 |
| mTAAR 8c | Novel | 1035 | — | — | 31 | 32 |
| mTAAR 9 | Novel | 1047 | — | — | 33 | 34 |

All genes for which discrepancies between published sequence data and the sequences provided by this invention were detected, as well as newly identified TAARs are underlined. Genbank accession numbers refers to published genes and the discrepancies between the published sequences and the sequences provided by the present invention. Cloning and sequence analysis of TAAR genes was carried out according to standard protocols employing genomic or cDNA from human (derived from total human brain total RNA, Stratagene). (nt=nucleotide sequence; aa=amino acid sequence).

Based on this sequence information the TAAR genes were amplified by means of PCR from either genomic DNA or cDNA of the respective species. Where not further specified, all procedures were carried out basically described in: Sambrook, J., Fritsch, E. F., und Maniatis, T. (1989). Molecular Cloning: A laboratory manual (New York: Cold Spring Harbor Laboratory Press). All reagents were of highest available purity, and all solutions and reagents were sterilized prior to use unless otherwise stated.

For this purpose oligonucleotide primers (table 2) were designed based on the TAAR coding sequences which had been derived from the genomic sequence information available at Genbank. The primers were designed such that the amplicons contain the entire open reading frames. The primer were designed using the VectorNTI software (Vector NTI version 9.0.0, Informax) following standard rules for PCR primers, mainly: a) Primers should have a length of 18-25 nt b) a G/C content of about 50% c) should not contain inverted repeats longer than 3 nt d) should carry an G or C at least at the 5′ end e) should have no simple repetitions of the same nts more than 3, and f) the annealing temperatures (TM) of primers used in the same PCR reaction should differ as little as possible (for details see for example: McPherson M. J., Hames B. D., Taylor G. R. (eds.): PCR 2—A Practical Approach. The practical approach series, Oxford University Press, 1995, ISBN: 0-19-963424-6). The annealing temperatures of the primer oligonucleotides were calculated based on rules described in: Breslauer K J, Frank R, Blocker H, Marky L A.: Predicting DNA duplex stability from the base sequence. Proc Natl Acad Sci U S A. 1986 June;83(11):3746-50 (These rules assume that the sequences are not symmetric and contain at least one G or C and have a length of minimally 8 nts). Oligonucleotides were ordered from Microsynth (Microsynth AG, Schützenstrasse 15, 9436 Balgach, Switzerland) in HPLC purified quality.

TABLE 2

Primer for cloning of mTAARs

| | | | | | |
|---|---|---|---|---|---|
| Receptor | Primer names | Sequence (5′→3′) | TM | SEQ. ID NO | Comment |

| | | | | | |
|---|---|---|---|---|---|
| mTAAR 2 | musTAR2_5_01 | atgaatacacccgacccc | 57.7 | 35 | |
| | musTAR2_3_01 | ctaaggatgtgcaggatgc | 56.4 | 36 | |
| mTAAR 2 | musGPR58_5_01 | atggcatcttttgaagcc | 55.8 | 37 | Cloned |
| | | | | | from |
| | | | | | cDNA |
| | musGPR58_3_01 | ctattctgtttctttttgagtaaacaa | 56.6 | 38 | |
| mTAAR 3 | musGPR57_5_01 | atggatctaatatacattcccgaag | 58.6 | 39 | |
| | musGPR57_3_01 | ttaatgtgcttcaggaaaaaga | 56.5 | 40 | |
| mTAAR 4 | musTAR2_5_01 | atgaatacacccgacccc | 57.7 | 41 | |
| | musTAR2_3_01 | ctaaggatgtgcaggatgc | 56.4 | 42 | |
| mTAAR 5 | musPNR_5_01 | atgagagctgtcctcctcc | 56.5 | 43 | |
| | musPNR_3_01 | tcagtcatggtataaatcaacagtc | 57.0 | 44 | |
| mTAAR 6 | musTAR4_5_01 | atgggcagtaactcgtctcc | 58.9 | 45 | |
| | musTAR4_3_01 | ttatatttgctcagagaacaagttg | 56.8 | 46 | |
| mTAAR 7a | musTAR8_5_01 | atggacaaattggttgacc | 55.2 | 47 | |
| | musTAR8_3_01 | ctactcaggaaacaagttggtg | 56.3 | 48 | |
| mTAAR 7b | musTAR12_5_01 | tggctacagataatgacagttttc | 57.3 | 49 | |
| | musTAR12_3_01 | ctactcaggaaacaagttggtg | 56.3 | 50 | |
| mTAAR | musTAR17_5_01 | atgactacaggtgatgacagttttc | 58.4 | 51 | |
| 7cΨ | musTAR17_3_01 | ctattcgggaaacaggttgg | 59.2 | 52 | |
| mTAAR 7d | musTAR14_5_01 | atggctacaggtgatgacag | 55.7 | 53 | |
| | musTAR14_3_01 | ctattcaggaaacaggttgg | 54.4 | 54 | |
| mTAAR 7e | musTAR9_5_01 | atggctacaggtgatgacag | 55.7 | 55 | |
| (TA9) | musTAR9_3_01 | ctattcaggaaacaggttgg | 54.4 | 56 | |
| mTAAR 7f | musTAR6_5_01 | atgtctatagctgatgaaactgtttc | 57.1 | 57 | |
| | musTAR6_3_01 | ctactcagaaaacagattggtgg | 57.7 | 58 | |
| mTAAR 8a | musTAR11_5_01 | atgaccagcaacttttccc | 57.3 | 59 | |
| | musTAR11_3_01 | ttactctgaaaataaatttgcagttg | 58.0 | 60 | |
| mTAAR 8b | musTAR7_5_01 | atgaccagcaacttttccc | 57.3 | 61 | |
| (TA7) | musTAR7_3_01 | ttactctgaaaacaaactcatggtag | 57.9 | 62 | |
| mTAAR 8c | musTAR10_5_01 | atgaccagcaacttttccc | 57.3 | 63 | |
| | musTAR10_3_01 | ttactctgaaaataaatttgtagttgat | 55.4 | 64 | |
| mTAAR 9 | musTAR3_5_01 | atgacaagcgacttctccc | 57.5 | 65 | |
| | musTAR3_3_01 | ttaacctgcacctgcctc | 57.0 | 66 | |

mTAAR2: 0The main reason of cloning the mTAAR 2 from cDNA was to assure whether the transcript of the corresponding genes would in fact contain the two exons. Independent from these experiments, the two coding exons have been cloned separately from genomic DNA. (Tm: melting temperature in ° C. calculated according to Breslauer K J, Frank R, Blocker H, Marky L A.: Predicting DNA duplex stability from the base sequence. Proc Natl Acad Sci U S A. 1986 June;83(11):3746-50; 5 in the primer name indicates a 5′ primer (i.e. musTAR3_{—}5_{—}01) and 3 in the primer name indicates a 3′ primer).

The actual PCR reactions had a total volume of 50 μl and the following composition: Template equaling 5-100 ng genomic DNA or cDNA equivalent to 100-200 ng of total RNA (sources specified below), 200 nM oligonucleotide primer, 1.5 mM MgCl₂ (Invitrogen), 200 mM dNTPs each (Invitrogen), lx concentrated PCR reaction buffer (Invitrogen) and 5 U/reaction recombinant Taq DNA polymerase (Invitrogen). The PCR reactions were assembled under a sterile working bench with UV irradiated equipment, and a) preparation of template material, b) assembly of PCR reactions, c) running the PCR reactions and agarose gel electrophoresis, and d) plasmid preparation were each performed in separated rooms in order to avoid any possible cross contamination of reagents with PCR amplified material or plasmid DNA. The PCR reactions were assembled in 200 μl PCR cups (Eppendorf) including Taq DNA polymerase at room temperature (RT) and transferred to the thermocycler (Geneamp 9700 with gold block, Applied Biosystems) preheated to 95° C. The temperature profile was adjusted as follows:

| | |
|---|---|
| 95° C. | 2 min |

(95° C.: 30 sec, annealing temperature: 30 sec,
72° C.: extension time) × cycle number:

| | |
|---|---|
| 72° C. | 5 min |
| 4° C. | max. 6 hrs |

annealing temperature: the Tm (melting temperature) of the oligonucleotide primer with the lower Tm value for the PCR reaction (calculated as specified above) −1° C. was used as annealing temperature. Example: 2 primers with Tm primer 1=55° C., Tm primer 2=57° C.→annealing temperature=54° C.

extension time was calculated by length of amplicon (in kb)×1 min.

cycle number: For each gene several PCR reactions were run in parallel with cycle numbers between 25-40. All PCR reactions were subsequently analyzed by agarose gel electrophoresis, and the PCR reaction with the lowest cycle number still producing a clearly visible PCR product of the correct size was used for subsequent cloning.

The PCR products were analyzed by agarose gel electrophoresis using 1% ultraPure agarose (GibcoBRL) made up in TAE buffer (Invitrogen). Agarose gels were run in PerfectBlue Horizontal Mini Electrophoresis Systems (Pequlab Biotechnologie GmbH, Erlangen, Germany) according to standard protocols (Sambrook et al., 1989). Agarose gels were stained with ethidium bromide, PCR products were visualized on a UV transilluminator (Syngene, Cambridge, UK), and the size of the PCR products was analyzed in comparison with the molecular weight standard 1 kb DNA ladder (Invitrogen). PCR products of the expected sizes were cut out from the gels with sterile scalpels (Bayha, Tuttlingen, Germany) and extracted from the agarose gel slices using the QIAquick Gel Extraction Kit (QIAGEN AG, Basel, Switzerland) following the instructions of the manufacturer.

The extracted PCR products were precipitated with a cold ethanol/sodium acetate precipitation (Sambrook et al., 1989), and DNA pellets were dissolved in a volume of 10 μl of 10 mM Tris/HCl pH 7.5. The PCR products in this solutions were subsequently cloned using the TOPO cloning kit for sequencing (Invitrogen; kits containing the vectors pCR4-TOPO and pCR2.1-TOPO were used) following the instructions of the manufacturer. Ligations were transformed into TOP 10 chemically competent bacteria (included in the TOPO cloning kit for sequencing) following the instructions of the manufacturer and subsequently plated on LB agar plates containing 100 μg/ml ampicillin (Sigma, Division of FLUKA Chemie GmbH, Buchs, Switzerland) and incubated at 37° C. over night. The bacterial colonies were analyzed by a method called “miniprep PCR”: Colonies were picked with a sterile inoculation loop (Copan Diagnostic S.P.A., Brescia, Italy) and transferred to new LB agar plates containing 100 μg/ml ampicillin. The same inoculation loops were subsequently transferred individually into 50 μl 10 mM Tris/HCl pH 7.5 in 1.5 ml Eppendorf Cups (Eppendorf) in order to transfer the bacteria which had remained on the loop into the solution. This bacterial suspensions were subsequently heated to 95° C. for 5 min, and 1 μl pf the resulting bacterial lysates per 50 μl PCR reaction was used as template for PCR reactions with primers flanking the multiple cloning site of the plasmids pCR4-TOPO and pCR2.1-TOPO (T7 and M13 reverse, sequences: Primer “T7”: 5′-cgggatatcactcagcataat-3′, primer “M13 reverse”: 5′-caggaaacagctatgacc-3′). PCR reactions were assembled as described above and run with the following temperature profile:

| | |
|---|---|
| 95° C. | 2 min |

(95° C.: 30 sec, 50° C.: 30 sec, 72° C.: 1 min) × 30 cycles

| | |
|---|---|
| 72° C. | 5 min |
| 4° C. | max. 6 hrs. |

The PCR products were analyzed by agarose gel electrophoresis as described above. Bacterial colonies for which cloned DNA fragments of the expected sizes could be detected by the method outlined above were used for subsequent plasmid preparation.

For plasmid preparations bacterial colonies identified to carry inserts of the expected sizes by “miniprep PCR” were used to inoculate bacterial liquid cultures in LB medium containing 100 μg/ml ampicillin, and cultures were incubated on a horizontal shaker at 37° C. over night (Sambrook et al., 1989). Plasmids were isolated from the bacterial liquid cultures using the HiSpeed Plasmid Maxi Kit (QIAGEN AG, Basel, Switzerland) following the instructions of the manufacturer. Plasmid concentrations were determined by measuring the OD at 260 nm using an UV/VIS spectrophotometer ultrospec 3300 pro (Amersham Biosciences Europe GmbH, Otelfingen, Switzerland). The size of inserts in the isolated plasmids was analyzed by means of restriction digests using an restriction endonuclease i.e. EcoRI (New England Biolabs, products distributed in Switzerland by Bioconcept, Allschwil, Switzerland); EcoRI restriction sites are flanking the multiple cloning sites in both vectors pCR4-TOPO and pCR2.1-TOPO, therefore the cloned inserts can be released from the plasmids by EcoRI restriction digests. Of each plasmid 0.5 μg were digested with EcoRI in a total volume of 20 μl following the recommendations of the manufacturer. Restriction digests were analyzed by agarose gel electrophoresis as described above. Plasmids which turned out to carry inserts of the expected sizes as revealed by restriction analysis were subjected to DNA sequence analysis, carried out by an external company (Microsynth A G, Schützenstrasse 15, 9436 Balgach, Switzerland).

The DNA sequences of the cloned TAAR gene open reading frames, as revealed by DNA sequence analysis, were compared to either previously published sequences and/or to the sequence of the TAAR genes retrieved from the genome sequence information. Potential errors in the DNA sequence information which could have been introduced by the PCR reaction were eliminated by comparing the sequences of the two to three DNA plasmids which were cloned each from independent PCR reactions per TAAR gene: Since the probability of the same PCR error to occur at the very same position in independent PCR reactions is basically zero, the alignment of the independent sequences for each gene revealed the very correct DNA sequence.

Template material: We paid particular attention to use standardized template material derived, were applicable, from the same inbred strains from which the genome sequence information available at Genbank was derived. Mice of the inbred strain C57BL/6, or genomic DNA derived from this mouse strain, was purchased from Jackson Laboratory (600 Main Street, Bar Harbor, Me. 04609 USA), rats of the inbred strain Crl:Wi was purchased from Charles River Laboratories France (Les Oncins, France; belongs to Charles River Laboratories, Inc.,251 Ballardvale Street, Wilmington, Mass. 01887-1000,USA). Genomic DNA of tail biopsies taken from adult Crl:Wi rats was prepared basically according to: Laird P W, Zijderveld A, Linders K, Rudnicki M A, Jaenisch R, Berns A.: Simplified mammalian DNA isolation procedure. Nucleic Acids Res. 1991 Aug. 11; 19(15):4293. The DNA concentration was determined by measuring the OD at 260 nm as described above and the DNA concentration was adjusted to 100 ng/μl. The DNA was stored in aliquots at 4° C. Human genomic DNA was purchased from Stratagene.

cDNAs were either commercially purchased (human: Human brain, cerebellum Marathon-Ready cDNA, BD Biosciences Clontech), or made from total RNA. RNA was either commercially purchased (human: human adult total brain, male, total RNA, Stratagene, Stratagene Europe, P.O. Box 12085 1100 AB Amsterdam, The Netherlands) or extracted from postnatal day 3 C57BL/6 mouse or Crl:Wi rat pups as described below.

RNA isolation: RNA was isolated from tissue samples basically according to: Chomczynski, P. und Sacchi, N.: Single-step method of RNA isolation by acid guanidinium thiocyanate-phenol-chloroform extraction. (Chomzynski P, Sacchi N., Analytical Biochemistry (1987) Single-Step Method of RNA Isolation by Acid Guanidinium Thiocyanate-Phenol-Chloroform Extraction 162, 156-159. Basic rules and principles for working with RNA are described in detail in Sambrook et al. (1989).

Rat or mice pups were killed by decapitation. The total brains were removed, weighed, and transferred to a 5 ml glass douncer and homogenized in the 10 volume of Trizol Reagent (Invitrogen). Total RNA was extracted from this homogenate following the instructions of the manufacturer. The RNA pellet derived from each half brain was dissolved on 200 μl 10 mM Tris/HCl pH 7.0. Traces of genomic DNA were removed by DNAseI digest (addition of 10 μl 100 μl MgCl₂ and 5 μl RNAse free DNAse I, 10³ U/μl, Roche; incubation at 37° C. for 1 hr). DNAse I was removed from the solution by phenol/chloroform extraction (H₂O saturated phenol pH 7.0, Sigma) according to Sambrook et al. (1989). The RNA was precipitated by cold ethanol/sodium acetate precipitate (Sambrook et al., 1989), dissolved in 10 mM Tris/HCl pH 7.0, and the RNA concentration was determined by measuring the OD at 260 nm as described above. This RNA preparation was used for the synthesis of cDNA.

cDNA synthesis: In an 1.5 ml Eppendorf cup was mixed: 2 μg total RNA in a total volume of 11 μl, 1 μl 0.5 μg/μl oligo dT12-18 (Invitrogen). The mixture was heated to 70° C. for 10 min, placed on ice, and the following reagents were added:

4 μl 5×reaction buffer (supplied with enzyme), 2 μl DTT (supplied with enzyme), 1 μl dNTPs (10 mM each, Amersham), 0.5 μl RNAse inhibitor RNAse-OUT (Invitrogen), 0.8 μl Superscript II Reverse Transcriptase (Invitrogen). The reaction was briefly vortexed, incubated for 1 hr at 42° C., the enzyme was inactivated by incubating at 70° C. for 10 min, and the reaction was adjusted to a final volume of 100 μl with 10 mM Tris/HCl pH 7.0. The cDNA was stored in aliquots at −80° C. prior to use.

In general, particular measures were taken to rule out sequence errors by the PCR reaction and by polymorphisms introduced by the template material: a) Cycle number: For each TAAR gene several PCR reactions with different cycle numbers were run, and the PCR reactions with lowest possible cycle number which still delivered a specific product in amounts sufficient for cloning were used. b) Several independent clones per gene: Each TAAR genes was cloned 2 or 3 times from independent PCR reactions, and the correct sequence was deduced from an alignment of the sequences of these independently generated plasmids: Since the likelihood of a PCR error to occur at the very same position in several independent PCR reactions is basically zero, the alignment of the sequence of these plasmids generates the very correct sequence of the gene. c) Standardized template material: Since differences between inbred strains can introduce significant inconsistencies in sequence information, we also paid particular attention in the choice of the template material employed for the cloning of TAAR genes: Mouse genomic DNA was purchased from Jackson Laboratory (600 Main Street, Bar Harbor, Me. 04609 USA) genomic DNA of the inbred strain C57BL/6 was used, since this strain was employed for the mouse genomic sequence database at Genbank), human genomic DNA was purchased from Stratagene, and rat genomic DNA was isolated from a rat of the inbred strain Crl:Wi purchased from Charles River Laboratories France (Les Oncins, France; belongs to Charles River Laboratories, Inc.,251 Ballardvale Street,Wilmington, Mass. 01887-1000,USA; the rat strain Crl:Wi was selected, since this strain was employed for the rat genomic sequence database at Genbank). For the synthesis of cDNA rat and mouse whole brain total RNA was isolated from a postnatal day 3 pup of either a Crl:Wi rat or a C57BL/6 mouse, employing the Trizol reagent following the recommendations of the manufacturer. Human total brain RNA was purchased from Stratagene. The cDNA was synthesized using SuperScript II Reverse Transcriptase from Invitrogen and dNTPs from Amersham following the recommendations of the manufacturers.

### Example 2

### Cell Culture & Generating Stable Cell Lines

Subcloning: For expression of TAAR receptors in mammalian cell lines, DNA fragments carrying the entire TAAR open reading frames were subcloned from the pCR4-TOPO or pCR2.1-TOPO vectors (Invitrogen) to pIRES-NEO2 (Rees S et al., Bicistronic vector for the creation of stable mammalian cell lines that predisposes all antibiotic-resistant cells to express recombinant protein. Biotechniques. 1996 January;20(1):102-4, 106, 108-10).

To this end, DNA fragments carrying the entire TAAR open reading frames were removed from the respective TOPO vectors by restriction digest with i.e. EcoRI and subsequent purification of the ˜1 kb fragments by agarose gel electrophoresis and gel extraction as described above. In parallel, the pIRES-NEO2 vector was linearized with EcoRI and dephosphorylated with shrimp alkaline phosphatase (Roche) following the instructions of the manufacturer. The DNA fragments carrying the entire TAAR open reading frames were ligated into the linearized pIRES-NEO2 vector using T4 DNA ligase (New England Biolabs) by mixing 30 ng of linearized pIRES-NEO2 vector, 100-300 ng of DNA fragments carrying the respective TAAR coding sequence, 1× ligation buffer (final concentration; provided with the enzyme as 10× concentrate) and 1 μl of T4 DNA ligase in a total volume of 20 μl. The reaction was proceeded at room temperature for 2-3 hrs, and the ligation was transformed into chemically competent TOP10 cells as described for the TOPO ligations. Plating of transformed bacteria and picking of bacterial clones was performed as described earlier for the PCR cloning of TAAR genes from genomic and cDNA. The picked bacterial clones were used to inoculate 5 ml liquid cultures in LB containing 100 μg/ml ampicillin. These liquid cultures were used for mini plasmid preparations using the QIAprep Miniprep Kit (QIAGEN), following the instructions of the manufacturer. The purified pIRES-NEO2 derived plasmid constructs were analyzed for the presence of inserts of the expected sizes by restriction analysis using EcoRI and agarose gel electrophoresis. Plasmids which were identified to carry inserts of the expected sizes were subjected to DNA sequence analysis carried out by Microsynth in order to make sure for the correct orientation and sequence of the inserts. Plasmids carrying the correct inserts in the correct orientation were used for expression of the respective TAARs in mammalian cell lines.

Cell culture: Basic cell culture handling and techniques used are described in Davis J M (ed.): Basic cell culture, sec. edition. Oxford University Press 2002, ISBN: 0199638535. TAARs were expressed in HEK cells (ATCC number: CRL-1573; described in Graham, F. L., Smiley, J., Russell, W. C., und Nairn, R. (1977). Characteristics of a human cell line transformed by DNA from human adenovirus type 5. Journal of General Virology 36, 59-74). The culture medium consisted of DMEM with Glutamax I, with sodium pyruvate, with pyridoxine, with 4500 mg/l glucose, Invitrogen Cat. # 31966-021; Penicillin/Streptomycin; fetal calf serum heat inactivated 10%. Cells were passaged at a ratio of 1:10-1:30 using Trypsin/EDTA (Invitrogen Cat. # 25300-062).

Stable cell lines: For the generation of stably transfected cell lines HEK cells were transfected with the pIRES-NEO2 expression vectors of the respective TAARs by using Lipofectamin 2000 transfection reagent and Optimem 1 reduced serum medium with Glutamax (Invitrogen Cat. # 51985-026) following the recommendations given in the Lipofectamine 2000 datasheet. After 24 hr post transfection cells were trypsinated and transferred into 90 mm tissue culture dishes (Nunc) at dilutions between 1:10-1:300 in culture medium supplemented with 1 mg/ml G418 which was renewed on a daily basis. After 7-10 days pot transfections well isolated clones were observed which were picked when they had reached a diameter of about 3 mm. Picking of clones was performed by detaching the entire clone from the culture dish using a 1 ml Gilson pipette, trypsinizing the clone in a 1.5 ml Eppendorf cup using Trypsin/EDTA (Invitrogen Cat. # 25300-062) and plating them into a 48 well dish, from where the cells were subsequently expanded by transferring them to increasing culture surface areas (48 well—12 well—60 mm etc.). Whenever cells were transferred to a new plate they were carefully trypsinized. As soon as single clones were selected, the concentration of G418 in the culture medium was reduced to 500 g/ml.

After sufficient expansion of the individual clones they were tested in a functional assay with the Amersham cAMP Biotrak Enzymeimmunoassay (EIA) System, using either the trace amines (p-tyramine, β-phenylethylamine (β-PEA), tryptamine, octopamine) or a selection of compounds (5-HT Serotonin, dopamine, norepinephrine and histamine). The success rate for generating stably transfected cell lines was about 1 out of 3 clones tested. This high success rate was made possible by the use of the pIRES vector, from which the TAARs are expressed in bicistronic transcripts carrying the TAAR CDSs and the NEO^{R} CDS in the same mRNA molecule.

TABLE 3
Pharmacology of mTAAR1. The amount of cAMP produced by HEK293
cells stably expressing the indicated receptor in response
to stimulation by each of the listed compounds was measured
using the cAMP Biotrak EIA System (Amersham). Shown are the
EC50 values in μM (means of at least three independent experiments;
EC50 values are calculated from experiments carried out with
12 concentrations each in duplicates evenly distributed in
the range of 30 μM-0.1 nM). The maximal response is represented
as percentage of the maximal cAMP levels induced by p-TYR.
In the experiments with dopamine the EC50 values were calculated
based on a maximal cAMP level of 50% compared to the level
reached by p-Tyramine.

| | Mouse TAAR 1 | |
|---|---|---|
| | EC₅₀(μM) | Max (96) |
| β-PEA | 0.66 +/− 0.21 | 100 |
| p-TYR | 1.37 +/− 0.33 | 100 |
| OCT | 19.71 +/− 4.78 | 100 |
| TRY | 1.99 +/− 0.38 | 100 |
| Dopamine | 11.76 +/− 5.43 | 50 |
| Serotonin, 5-HT | >50.000 | |
| Norepinephrine | >50.000 | |
| Histamine | >50.000 | |
| N-methyl-β-PEA | 0.15 +/− 0.03 | 100 |
| N-methyl-p-tyramine | 1.02 +/− 0.39 | 100 |

For all other mTAARs than mTAAR 1 a minimum of 10 independent clones were tested. If no responder to either trace amines or selected top compounds could be detected it was concluded that the respective TAAR was not responsive to the compounds at all under the defined test conditions.

cAMP assay: cAMP was assayed using the cAMP Biotrak Enzymeimmunoassay (EIA) System from Amersham as described in the product manual, following the so-called Non-acetylation EIA procdure. The general procedure is also described i.e. in Notley-McRobb L et al. (The relationship between external glucose concentration and cAMP levels inside Escherichia coli: implications for models of phosphotransferase-mediated regulation of adenylate cyclase. Microbiology. 1997 June;143 (Pt 6):1909-18) and Alfonso A, de la Rosa L et al. (Yessotoxin, a novel phycotoxin, activates phosphodiesterase activity. Effect of yessotoxin on cAMP levels in human lymphocytes. Biochem Pharmacol. 2003 Jan. 15;65(2):193-208.)

In the protocol the following minor modifications were introduced: After stimulation of HEK cells in 96 well plates the reaction was stopped by applying equal volume of pre cooled (−20° C.) ethanol. Lysis of cells was not achieved by applying the lysis buffer supplied with the kit, but with transferring the 96 well plates to −80° C. for a minimum of 6 hrs (up to several days). Thereafter the contents of the 96 well plates was dried at 50° C. over night, and the material remaining in the 96 well plates was resuspended in 200 μl of the assay buffer (the buffer used for resuspending all reagents of the kit). The resuspension were assayed in serial dilutions in assay buffer.

Phylogenetic Analysis

The human, mouse and rat genome sequences (human: NCBI reference draft 34, July 2003; mouse: NCBI draft 32, October 2003; rat: Rat Genome Sequencing Consortium draft 3, June 2003) were screened, employing an in-house genomic sequence analysis resource for the identification of all TAAR genes and in particular for the presence of previously unknown potential members of the TAAR family. This screen led to the identification of 44 TAAR genes overall in human, mouse and rat, among them 22 novel genes (including 2 pseudogenes and a human TAAR 7 gene fragment) and 11 genes with discrepancies to previously published sequences. The comparison of simple DNA and protein sequence, as well as of more complex parameters like the GPCR pharmacophore relationship of the ligand pocket vectors and the phylogenetic relationship of the TAAR genes with the genome sequences strongly support the conclusion that the TAAR receptor listed in FIG. 3 represents the complete TAAR receptor family in human, mouse and rat. The identification of TAAR genes based on the genome sequence information was largely facilitated by the fact that all TAARs except TAAR 2 are encoded by single exons..

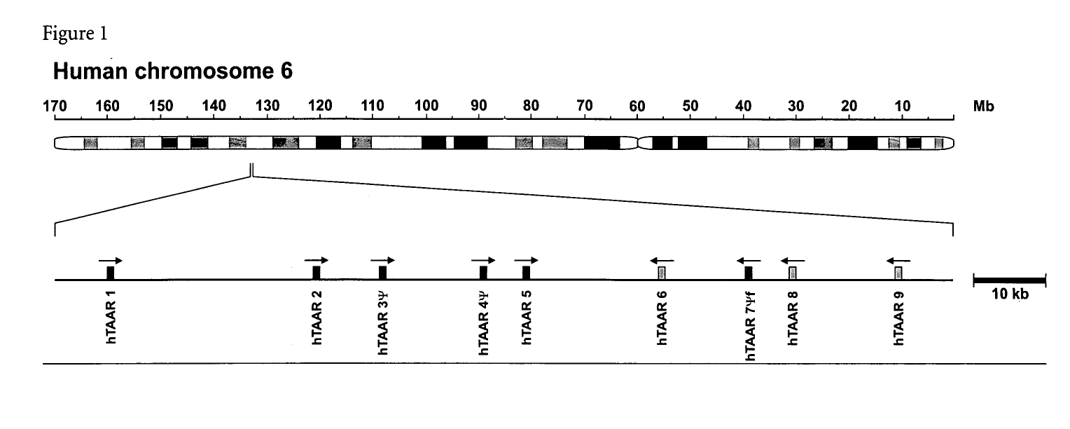

In this context the arrangements of the TAAR genes in the human, mouse and rat genomes were analyzed (for human see FIG. 1). The genes mapped to very compact regions spanning a total of about 109 kb in human (chromosome 6q23.1), 192 kb in mouse (chromosome 10A4) and 216 kb in rat (chromosome 1p12; all chromosomal regions as assigned by LocusLink), with no other annotated or predicted genes in the same chromosomal regions. With the exception of TAAR 2, which is encoded by two exons, the coding sequences of all TAAR genes are located in a single exon and are very similar in their length of about 1 kb throughout all three species (range: 999-1089 bp). It is interesting to note that the region 6q23.1 on human chromosome no. 6 is known as schizophrenia susceptibility locus (Levinson et al.,: Multicenter linkage study of schizophrenia candidate regions on chromosomes 5q, 6q, 10p, and 13q: schizophrenia linkage collaborative group III. Am J Hum Genet. 2000 September;67(3):652-63. Epub 2000 Aug. 2.; Martinez et al., Follow-up study on a susceptibility locus for schizophrenia on chromosome 6q. Am J Med Genet. 1999 Aug. 20;88(4):337-43.) which underlines the potential of TAARs as drug targets for psychiatric disorders.

In human TAAR 1-5, as well as TAAR 6, 8 and 9 were found to be arranged in two blocks of genes with an inverse orientation of the open reading frames. The most striking difference between human and rodents is the complete absence of any functional TAAR 7 paralogues from the human genome, which represent almost half of the entire TAAR family in rat and for which we detected only a degenerate gene fragment in human with closest similarity to rTAAR 7h. In addition, TAAR 3 and 4 are functional receptors in rodents, but pseudogenes in human. The pseudogenization of hTAAR 3Ψ and 4Ψ likely is a recent event, since these genes are overall very well preserved with only two and three bp changes in the coding sequences, respectively, rendering them non-functional. The different total number of nine TAAR 7 paralogues in rat (including two pseudogenes, rTAAR 7fΨ and rTAAR 7iΨ) and six TAAR 7 paralogues in mouse (including one pseudogene, mTAAR 7cΨ) represents a remarkable inter-rodent difference within a family of such closely related genes, which is reflected in strong predicted ligand binding differences between individual mouse and rat TAARs determined by the GPCR pharmacophore relationship of the ligand pocket vector analysis (see below). The strong differences detected in the predicted ligand binding pockets of receptors with highly similar overall protein sequence indicates that the overall amino acid sequence identity is no suitable measure for predicting the degree of similarity of mouse and rat TAARs with regard to their pharmacological profile, a fact requiring close attention for the development of behavioral animal models.

Triggered by the compact chromosomal arrangement of TAAR genes in human and rodents and the high number of very similar genes within each species, the phylogenetic relationship of TAAR genes was analyzed across all three species. The phylogenetic analysis shows that the TAAR genes originate in a putative common ancestor from which they evolved through a total of eight gene duplication events, leading to a set of nine genes before the primate and rodent lineage split (FIG. 2; gene duplication events from the common ancestor, speciations leading to human and primate lineage, and gene duplications within the rodent lineage are each collectively indicated by numbers 1, 2 and 3, respectively). In seven cases, these genes were further duplicated into orthologeous sets of three by the two speciation events leading to human, mouse and rat. The rodent TAAR 7 and 8 orthologues evolved by further, independent duplications within the rat and mouse lineages. It is important to note that these events can only have occurred in the sequential order outlined above.

The overall structure of the phylogenetic tree immediately suggested to us the distinction of the TAAR family into three subgroups (indicated by grey boxes in FIG. 3). As discussed in detail below it turned out that the concept of subgroups is not an arbitrary distinction inferred just by the overall DNA sequences, but it reflects significant functional differences.

There are a total number of five pseudogenes in human, rat and mouse. All pseudogenes could be transformed into functional proteins by introduction of minor changes. Following a description of the most obvious and likely mutational events that might have caused the pseudogenizations: hTAAR 3Ψ became a pseudogene most likely due to a deletion of two bps (CA) at position 135 causing an early termination. A pointmutation (A411T) turning a Cys into a stop codon and two insertions (G605, A750) might have caused the pseudogenisation of hTAAR 4Ψ. rTAAR 7fΨ has two obvious differences to all the other TAAR 7 members of 12 additional bps at position 726, probably caused by an insertion event and of a pointmutation (A515G) turning a Trp into a stop codon. The second rat pseudogene rTAAR 7iΨ misses 10 bps at position 90 causing an early termination, and mTAAR 7cΨ misses 15 bps at position 895 (intracellular loop 3) and 7 bps at position 513 causing an early termination. In summary, within the TAAR family pseudogenisation seems to be caused by all possible mutational events like pointmutations, deletion and insertions. The events seem to have occurred fairly recently due to the highly conserved overall sequence. The pharmacology of such repaired pseudogenes might give insights into the importance of TAARs in the context of adaptation processes during evolution.

The foregoing description illustrates and describes the invention of the present disclosure. Additionally, the disclosure shows and describes only certain embodiments of the invention but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form(s) disclosed herein.

1. An isolated or recombinant polypeptide comprising SEQ ID NO: 5.

2. An isolated or recombinant polypeptide comprising SEQ ID NO: 7

3. An isolated or recombinant polypeptide comprising SEQ ID NO: 9.

4. An isolated or recombinant polypeptide comprising SEQ ID NO: 11.

5. An isolated or recombinant polypeptide comprising SEQ ID NO: 13.

6. An isolated or recombinant polypeptide comprising SEQ ID NO: 15.

7. An isolated or recombinant polypeptide comprising SEQ ID NO: 17.

8. An isolated or recombinant polypeptide comprising SEQ ID NO: 22.

9. An isolated or recombinant polypeptide comprising SEQ ID NO: 24.

10. An isolated or recombinant polypeptide comprising SEQ ID NO: 26.

11. An isolated or recombinant polypeptide comprising SEQ ID NO: 28.

12. An isolated or recombinant polypeptide comprising SEQ ID NO: 30.

13. An isolated or recombinant polypeptide comprising SEQ ID NO: 32.

14. An isolated or recombinant polypeptide comprising SEQ ID NO: 34.

15. An isolated or recombinant polynucleotide comprising SEQ. ID NO: 4.

16. An isolated or recombinant polynucleotide comprising SEQ. ID NO: 6.

17. An isolated or recombinant polynucleotide comprising SEQ. ID NO: 8.

18. An isolated or recombinant polynucleotide comprising SEQ. ID NO: 10.

19. An isolated or recombinant polynucleotide comprising SEQ. ID NO: 12.

20. An isolated or recombinant polynucleotide comprising SEQ. ID NO: 14.

21. An isolated or recombinant polynucleotide comprising SEQ. ID NO: 16.

22. An isolated or recombinant polynucleotide comprising SEQ. ID NO: 21.

23. An isolated or recombinant polynucleotide comprising SEQ. ID NO: 23.

24. An isolated or recombinant polynucleotide comprising SEQ. ID NO: 25.

25. An isolated or recombinant polynucleotide comprising SEQ. ID NO: 27.

26. An isolated or recombinant polynucleotide comprising SEQ. ID NO: 29.

27. An isolated or recombinant polynucleotide comprising SEQ. ID NO: 31.

28. An isolated or recombinant polynucleotide comprising SEQ. ID NO: 33.

29. A vector comprising the polynucleotide selected from one of the group consisting of SEQ. ID NO: 4, SEQ. ID NO: 6, SEQ. ID NO: 8, SEQ. ID NO: 10, SEQ. ID NO: 12, SEQ. ID NO: 14, SEQ. ID NO: 16, SEQ. ID NO: 21, SEQ. ID NO: 23, SEQ. ID NO: 25, SEQ. ID NO: 27, SEQ. ID NO: 29, SEQ. ID NO: 31, SEQ. ID NO: 33.

30. A host cell comprising the vector according to claim 29.

31. A transgenic non-human animal comprising the polynucleotide according to 30.

32. A fingerprint motif comprising the sequence NSXXNPXXZXXXBXWF (SEQ ID NO: 1) wherein X is any natural occurring amino acid, Z is selected from the group consisting of tyrosine or histidine and B is selected from the group consisting of tyrosine or phenylalanine.

33. A method for identifying mouse TAARs comprising comparing the fingerprint motif comprising the sequence NSXXNPXXZXXXBXWF (SEQ ID NO: 1) wherein X is any natural occurring amino acid, Z is selected from the group consisting of tyrosine or histidine and B is selected from the group consisting of tyrosine or phenylalanine with a polypeptide sequence, wherein a 100% identity of the fingerprint sequence with the polypeptide sequence identifies mouse TAARs within the polypeptide sequence.

34. An isolated or recombinant polypeptide having a sequence which comprises the fingerprint motif comprising the sequence NSXXNPXXZXXXBXWF (SEQ ID NO: 1) wherein X is any natural occurring amino acid, Z is selected from the group consisting of tyrosine or histidine and B is selected from the group consisting of tyrosine or phenylalanine and differs from the sequences SEQ. ID NOs: 3.

35. A method for identifying compounds which bind to a polypeptide of the invention (polypeptide D) comprising:
a) contacting the polypeptide, wherein the polypeptide D is selected from the group consisting of SEQ ID NOs 5, 7, 9,11, 13, 15, 17, 22, 24, 26, 28, 30, 32, 34 or the sequence NSXXNPXXZXXXBXWF (SEQ ID NO: 1), wherein X is any natural occurring amino acid, Z is selected from the group consisting of tyrosine or histidine and B is selected from the group consisting of tyrosine or phenylalanine, with a candidate compound and
b) determining whether said candidate compound binds to said polypeptide.

36. A method for identifying compounds which modulate the biological activity of a polypeptide of the invention (polypeptide D) comprising
a) contacting a polypeptide wherein the polypeptide D is selected from the group consisting of SEQ ID NOs 5, 7, 9, 11, 13, 15, 17, 22, 24, 26, 28, 30, 32, 34 or the sequence NSXXNPXXZXXXBXWF (SEQ ID NO: 1), wherein X is any natural occurring amino acid, Z is selected from the group consisting of tyrosine or histidine and B is selected from the group consisting of tyrosine or phenylalanine, with a candidate compound and
b) determining if said candidate compound has modulated the function or activity of said polypeptide.


SEQUENCE LISTING (136 sequences)

SEQ ID NO: 1 - protein, 16 aa, Mus musculus
  MOD_RES (3)..(4): Variable amino acid
NSXXNPXXXXXXXXWF

SEQ ID NO: 2 - DNA, 999 bp, Mus musculus
  mTAAR1
atgcatctttgccacgctatcacaaacatttcccacagaaacagcgactggtcaagagaagtccaagcttccctgtacagcttaatgtcactcataatcctggccactctggttggcaacttaatagtaataatttccatatcccatttcaagcaacttcatacacccaccaactggctccttcactccatggccattgtcgactttctgctgggctgtctgataatgccctgcagcatggtgagaactgttgagcgctgttggtattttggggaaatcctctgtaaagttcacaccagcaccgatatcatgctgagctccgcctccattttccacttagctttcatttccattgaccgctactgtgctgtgtgtgaccctttgagatacaaagccaagatcaatatctccactattcttgtgatgatcctcgttagttggagccttcctgctgtttatgcatttgggatgatcttcctggaactgaacttaaaaggagtggaagagctgtatcgcagtcaggtcagcgacctgggcggctgttctcccttctttagtaaagtatctggggtactggcgttcatgacttccttctatatacctggatctgttatgttatttgtttactataggatatatttcatagctaaaggacaagcaaggtcaatcaatcgtacgaatgttcaagttggattggaagggaaaagccaagcaccacaaagcaaggaaacaaaagccgcgaagaccttagggatcatggtgggcgttttcctcgtatgctggtgcccgttctttctctgcacggtcctggaccctttcctgggctatgttatcccaccctctctgaatgacgcactgtattggtttgggtacttgaattctgccctcaatccgatggtttatgcctttttctatccctggttcagaagagccttgaagatggttctccttggtaaaattttccaaaaagattcatctaggtctaagctatttttgtaa

SEQ ID NO: 3 - protein, 332 aa, Mus musculus
  mTAAR1
MHLCHAITNISHRNSDWSREVQASLYSLMSLIILATLVGNLIVIISISHFKQLHTPTNWLLHSMAIVDFLLGCLIMPCSMVRTVERCWYFGEILCKVHTSTDIMLSSASIFHLAFISIDRYCAVCDPLRYKAKINISTILVMILVSWSLPAVYAFGMIFLELNLKGVEELYRSQVSDLGGCSPFFSKVSGVLAFMTSFYIPGSVMLFVYYRIYFIAKGQARSINRTNVQVGLEGKSQAPQSKETKAAKTLGIMVGVFLVCWCPFFLCTVLDPFLGYVIPPSLNDALYWFGYLNSALNPMVYAFFYPWFRRALKMVLLGKIFQKDSSRSKLFL

SEQ ID NO: 4 - DNA, 1020 bp, Mus musculus
  mTAAR2
atggcatcttttgaagcccagcaggaaacctttgattgttcagagtacggaaatggatcttgcccagagaatgaaagatctctcggcgtccgagctgccatgtactcgttgatggcctgcgccatattcatcacgatctttggcaacctggccatgatcatttccatttcctacttcaaacagcttcacacaccgaccaacctcctcattctctccatggccgtcaccgacttccttctcggattcaccatcatgccatatagtatggtcaggtcagtggaaaactgctggtatttcggacttacgttttgcaagatccattatagcttcgacctgatgcttagcataacgtccattttccacctttgctcggtggccgtcgatagattttatgccatctgtcaccctttgcattattgcaccaaaatgactatcccggtcgttaggcggctgctgctcgtctgctggtcagtccccggggcgtttgcctttggggtggtcttctccgaggcttacgctgatggaattgaaggctatgacattttggttgcatgttccagttcctgcccagtcatgttcaacaagctatgggggaccaccttgtttgtggcaggctttttcactcctagctcgatgatggtggggatttacggcaaaattttcgctgtatccaaaaaacacgctcgcgtaattgacaacttgccagaaaatcaaaacaatcaaatgaggaaggacaaaaaagcagccaaaactttagggatagtgatgggtgttttcttattgtgttggtttccatgtttcttcaccatcctgttagatccgtttctgaatttctctacgcctgcagttctgtttgatgccttaacatggttcggttattttaattccacatgtaatcccttaatttatggtttcttctacccttggtttcgcagagcactcaagtacattttactgggtaaaattttcagctcgcatttccataatactaatttgtttactcaaaaagaaacagaatag

SEQ ID NO: 5 - protein, 339 aa, Mus musculus
  mTAAR2
MASFEAQQETFDCSEYGNGSCPENERSLGVRAAMYSLMACAIFITIFGNLAMIISISYFKQLHTPTNLLILSMAVTDFLLGFTIMPYSMVRSVENCWYFGLTFCKIHYSFDLMLSITSIFHLCSVAVDRFYAICHPLHYCTKMTIPVVRRLLLVCWSVPGAFAFGVVFSEAYADGIEGYDILVACSSSCPVMFNKLWGTTLFVAGFFTPSSMMVGIYGKIFAVSKKHARVIDNLPENQNNQMRKDKKAAKTLGIVMGVFLLCWFPCFFTILLDPFLNFSTPAVLFDALTWFGYFNSTCNPLIYGFFYPWFRRALKYILLGKIFSSHFHNTNLFTQKETE

SEQ ID NO: 6 - DNA, 1032 bp, Mus musculus
  mTAAR3
atggatctaatatacattcccgaagacttatccagctgtccaaaatttggaaataaatcctgccctcccaccaatcgctctttccgtgtccgcatgataatgtacctgtttatgactggagccatggttatcaccatctttggaaacttagtgataatcatttccatatcgcatttcaaacagctacattctcccaccaactttctgatcctctccatggcaaccacggacttcctgctgggttttgtcatcatgccttacagtatggtgagatcagtggagagctgctggtatttcggagacagcttttgcaaattccacgcgagcttcgacatgatgctaagcctgacgtccatattccacctgtgctccatcgcgatcgaccggttttatgcagtgtgtgaccctttgcactacaccaccaccatgacggtatccatgatcaagagactgctggctttttgctgggcagccccagctctcttttcgtttggtttagttctgtcagaggccaatgtttctggtatgcagagctacgagattcttgttgcttgcttcaatttctgtgcgcttacgttcaacaagttttgggggaccatactgttcactacctgcttcttcactcctggctccatcatggttggtatttatggtaaaatttttattgtttctagacgacatgctcgagccctcagcgatatgcctgcgaacacaaaaggagcagtagggaaaaacctgtctaagaaaaaggacaggaaagcagctaagaccctgggcatcgtcatgggggtgtttctagcgtgctggctgccttgcttcctggctgttttgattgatccatatttagactactccacgcccatcatagtgctggatcttctggtatggctcgggtacttcaactctacttgcaaccccctcatccatggctttttttacccgtggtttcggaaagctcttcagtttatagtgtcaggaaaaatatttcgctctaattcagacactgcaaatctttttcctgaagcacattaa

SEQ ID NO: 7 - protein, 343 aa, Mus musculus
  mTAAR3
MDLIYIPEDLSSCPKFGNKSCPPTNRSFRVRMIMYLFMTGAMVITIFGNLVIIISISHFKQLHSPTNFLILSMATTDFLLGFVIMPYSMVRSVESCWYFGDSFCKFHASFDMMLSLTSIFHLCSIAIDRFYAVCDPLHYTTTMTVSMIKRLLAFCWAAPALFSFGLVLSEANVSGMQSYEILVACFNFCALTFNKFWGTILFTTCFFTPGSIMVGIYGKIFIVSRRHARALSDMPANTKGAVGKNLSKKKDRKAAKTLGIVMGVFLACWLPCFLAVLIDPYLDYSTPIIVLDLLVWLGYFNSTCNPLIHGFFYPWFRKALQFIVSGKIFRSNSDTANLFPEAH

SEQ ID NO: 8 - DNA, 1044 bp, Mus musculus
  mTAAR4
atgaatacacccgacccctggagctccccagaagtacagttttgctttgctgctgcaaacagttcttgcccaaggaaggcgaggcctgcgctcgttgtctgtgccatgtacctcatcatgattggagcgatagtgatgaccatgctgggaaacatggctgtgatcatctctattgcccacttcaagcagctccactccccaaccaacttccttattctctccatggctaccacagacttcctgttgagttgcgtggtcatgcccttcagtatgatccggtccatcgagtcatgctggtacttcggagacctcttttgcaaagttcacagctgctgtgacatcatgctctgtaccacatccattttccacctctgcttcatctcagttgaccgccactatgccgtctgcgaccctttgcactacgtcacccaaatcacgacccgggtggtaggggtctttctactcatcagttggtctgttcccatcttttttgcctttggcttggtattctcagaattaaatctgattggtgctgaggattttgtcgcagccattgactgtacaggtttgtgtgtgttgatatttaacaagctctggggagttctggcttccttcatagctttctttctgcctgggacagtcatggtagggatttacatacacattttcacagttgcccagaaacatgccaggcagattggtacaggtcctaggacaaaacaggccctctcagaaagcaaaatgaaggccacatcaaaaaaggaaagcaaggccaccaagactttaagcattgtcatgggagtgtttgtgttgtgttggctacccttttttgtcttgacaatcacagaccctttcattgattttacaacccctgaagatttgtataatgttttcctttggcttggttattttaattccactttcaatcccatcatatatggcatgttctatccctggtttcgaaaggccctgaggatgatagtcacaggaacgatcttccgctctgactcttctacctcaagcctgcatcctgcacatccttag

SEQ ID NO: 9 - protein, 347 aa, Mus musculus
  mTAAR4
MNTPDPWSSPEVQFCFAAANSSCPRKARPALVVCAMYLIMIGAIVMTMLGNMAVIISIAHFKQLHSPTNFLILSMATTDFLLSCVVMPFSMIRSIESCWYFGDLFCKVHSCCDIMLCTTSIFHLCFISVDRHYAVCDPLHYVTQITTRVVGVFLLISWSVPIFFAFGLVFSELNLIGAEDFVAAIDCTGLCVLIFNKLWGVLASFIAFFLPGTVMVGIYIHIFTVAQKHARQIGTGPRTKQALSESKMKATSKKESKATKTLSIVMGVFVLCWLPFFVLTITDPFIDFTTPEDLYNVFLWLGYFNSTFNPIIYGMFYPWFRKALRMIVTGTIFRSDSSTSSLHPAHP

SEQ ID NO: 10 - DNA, 1014 bp, Mus musculus
  mTAAR5
atgagagctgtcctcctcccgggctctggagagcagcctacagcgttctgctaccaggtgaatgggtcttgccccaggacagtccacccgctggccatccaggtcgtcatctatctggcctgcgcagtgggtgtgctgatcacagtcctggggaatttgtttgtggtgtttgcagtgtcctactttaaagtgctccacacgcccaccaacttcctgctgctctccctggccctggcagacatgttgctaggtctgctggtgctgcccctgagcacagtccgctctgtggagagctgctggttctttggagacttcctgtgccgtctgcatacctatctggacacgctgttctgtctcacctccatcttccatctctgttttatttccattgaccgtcattgtgccatctgtgaccccctgctctatccctccaagttcacagtcaggacagccctccgctacatcgtggcagggtgggggataccagcagcctacactgccttcttcctctacacagatgtggtagagagagcactcagccagtggctagaagagatgccttgtgtgggcagctgccagctgctgttcaataagttctggggttggttaaacttccctgcattttttgttccctgcctaatcatgatcagcttgtacctgaagatctttgtggtcgccaccaggcaggctcagcagatcagaactctgagccaaagcttggctggggctgtcaagcgggaaagaaaagctgccaagacgctgggcattgctgtgggcatctaccttgtgtgttggcttcccttcactgttgacacactagtggacagccttcttaacttcatcaccccacccctggtctttgacatctttatctggtttgcttatttcaattcagcctgtaatcccatcatctacgtcttctcctaccggtggtttaggaaggcactgaaactgctcctgagccgggagatcttctcaccaaggacaccgactgttgatttataccatgactga

SEQ ID NO: 11 - protein, 337 aa, Mus musculus
  mTAAR5
MRAVLLPGSGEQPTAFCYQVNGSCPRTVHPLAIQVVIYLACAVGVLITVLGNLFVVFAVSYFKVLHTPTNFLLLSLALADMLLGLLVLPLSTVRSVESCWFFGDFLCRLHTYLDTLFCLTSIFHLCFISIDRHCAICDPLLYPSKFTVRTALRYIVAGWGIPAAYTAFFLYTDVVERALSQWLEEMPCVGSCQLLFNKFWGWLNFPAFFVPCLIMISLYLKIFVVATRQAQQIRTLSQSLAGAVKRERKAAKTLGIAVGIYLVCWLPFTVDTLVDSLLNFITPPLVFDIFIWFAYFNSACNPIIYVFSYRWFRKALKLLLSREIFSPRTPTVDLYHD

SEQ ID NO: 12 - DNA, 1038 bp, Mus musculus
  mTAAR6
atgggcagtaactcgtctccgcccaccgtcctgcagctctgctatgagaatgtgaccgggtcttgtgtgaaaaccccctactctcctgggtcgagggtcatcctctatgcagtctttggctttggggctgtgctggcagtgtttggaaacctcatggtgatgatatccatcctccatttcaagcagctgcactctcccaccaatttcctcattgcatccttggcctgtgctgactttggtgtgggcatctctgtgatgcccttcagcatggtcaggtccatcgagagctgctggtactttggtagaagtttctgcactttccacacctgctgtgatgtggcgttctgttactcttctctcttccacctgagcttcatctccatcgacaggtacatcgctgtcacggaccctctggtctatcccaccaagttcacggtgtctgtgtctggaatttgcatcggcgtctcctggattctgcccctggtgtacagcggcgctgtgttctacacaggggtttatgatgacggcctggaggaattatccagtgccctcaactgcgtagggggctgtcaggtagttgtgaatcagaactgggtgctgatagattttctttccttcttaatacctacacttgttatgataattctgtatggtaatatttttctggtagctagacaacaggctaaaaagatagaaaatattgggagcaaaacagaatcatcctctgagagctacaaagccagagtggcgagaagagaaagaaaagcagccaaaaccctggggattaccgtggtagcattcatgatatcatggttgccctacagcattgattcattagttgatgcttttatgggcttcatcacccctgcctacatttatgaaatctgtgtttggtgtgcttattacaactcagccatgaaccctctgatctacgctttattttatccatggtttaagaaagctattaaggttattatgagtggccaggtgttcaagaacagctcagccactatgaacttgttctctgagcaaatataa

SEQ ID NO: 13 - protein, 345 aa, Mus musculus
  mTAAR6
MGSNSSPPTVLQLCYENVTGSCVKTPYSPGSRVILYAVFGFGAVLAVFGNLMVMISILHFKQLHSPTNFLIASLACADFGVGISVMPFSMVRSIESCWYFGRSFCTFHTCCDVAFCYSSLFHLSFISIDRYIAVTDPLVYPTKFTVSVSGICIGVSWILPLVYSGAVFYTGVYDDGLEELSSALNCVGGCQVVVNQNWVLIDFLSFLIPTLVMIILYGNIFLVARQQAKKIENIGSKTESSSESYKARVARRERKAAKTLGITVVAFMISWLPYSIDSLVDAFMGFITPAYIYEICVWCAYYNSAMNPLIYALFYPWFKKAIKVIMSGQVFKNSSATMNLFSEQI

SEQ ID NO: 14 - DNA, 1077 bp, Mus musculus
  mTAAR7a
atggacaaattggttgaccattttctcagtgatcaatctagaaccatgaatgaagatctgttctctgccacatctacagagctgtgctatgagaacctgaacaggtcctgtgtcaggagcccatactccccaggccctcgcctcatcctctatgcagtctttggctttggggctgcgctggctgtgtgtggaaacctcctggtgatgacatcaattcttcatttcaggcagctgcactctcctgccaacttcctggtggcatccctggcctgtgctgatttcttagtgggtctgactgtgatgcccttcagcacggtgaggtctgtggagggctgctggtactttggagagagttattgtaaattccattcttgttttgaagggtcattctgttactcttctatcttccacttgtgcttcatctctgttgatagatatattgcagtcagtgaccccctgacctaccccaccaggttcactgcttctgtttctggcaagtgcatcaccttctcctggctcctgtccatcatctacagcttttccctcctttacacaggagccaacgaagctgggctggaggatctagtgagtgtcctcacctgtgtgggtggctgtcaaattgcagtgaatcaaagctgggtctttatcaactttctattatttcttatccccactcttgtgatgatgactgtctactctaagattttcctcattgctaaacagcaggctcagaacattgagaagatgagcaagcagactgccagggcatcagaaagctacaaggacagggtggccaagagggagaggaaagcagccaaaaccctgggcattgcagtggctgccttcctcctttcatggctgccatacttcattgactccatcattgatgccttcctagggttcatcacgcccacgtatgtgtatgaaatcctagtttggatcgcttactataactcagccatgaaccctttgatttatgctttcttttatccttggtttcgaaaagccatcaagctcattgtcactggcaaaatcttgagggagaattcctcaaccaccaacttgtttcctgagtag

SEQ ID NO: 15 - protein, 358 aa, Mus musculus
  mTAAR7a
MDKLVDHFLSDQSRTMNEDLFSATSTELCYENLNRSCVRSPYSPGPRLILYAVFGFGAALAVCGNLLVMTSILHFRQLHSPANFLVASLACADFLVGLTVMPFSTVRSVEGCWYFGESYCKFHSCFEGSFCYSSIFHLCFISVDRYIAVSDPLTYPTRFTASVSGKCITFSWLLSIIYSFSLLYTGANEAGLEDLVSVLTCVGGCQIAVNQSWVFINFLLFLIPTLVMMTVYSKIFLIAKQQAQNIEKMSKQTARASESYKDRVAKRERKAAKTLGIAVAAFLLSWLPYFIDSIIDAFLGFITPTYVYEILVWIAYYNSAMNPLIYAFFYPWFRKAIKLIVTGKILRENSSTTNLFPE

SEQ ID NO: 16 - DNA, 1077 bp, Mus musculus
  mTAAR7b
atggctacagataatgacagttttccctgggatcaagacagcatcctgagcagtgatatgttctctgccacatctacagagctgtgctatgagaacctgaacaggtcctgtgtcaggagcccatattccccaggccctcgcctcatcctctatgcagtctttggctttggggctgcgctggctgtgtgtggaaacctcctggtgatgacatcaattcttcatttcaggcagctacactcccctgccaacttcctggtggtgtccttggcctgtgctgatttcttggtgggattgactgtgatgcccttcagcacagtgaggtctgtggagggatgctggtactttggggagagttactgtaaattacacacttgttttgatgtatctttctgctattgttctatcttccacttgtgcttcatctctgttgatagatatattgcagtcagtgaccccctgacctaccccaccaggtttactgcatttgtttctggcaagtgcatcaccttctcctggctcctgtccaccatttatgggttttctctcctttatacaggggctaatgaagctgggctggaggatctagtgagtgccctcacctgtgtgggtggctgtcaacttgcagtgaatcaaagctgggtcttcatcaatttcctattatttcttatccccacccttgtgatgataactgtctactctaagattttcctcattgctaaacagcaggctcagaacattgagaagatgagcaagcagactgccagggcatcagacagctacaaggacagggtggccaagagagagaggaaagcagccaaaaccctgggcatcgcagtggctgccttcctcctttcatggctgccatacttcattgactccatcattgatgccttcctagggttcatcacgcccacgtatgtgtatgaaatcctagtttggatcgcttactataactcagccatgaaccctttgatttatgctttcttttatccttggtttcgaaaagccatcaaactcattgtctctggcaaagtcttgagggagaattcctcaaccaccaacttgtttcctgagtag

SEQ ID NO: 17 - protein, 358 aa, Mus musculus
  mTAAR7b
MATDNDSFPWDQDSILSSDMFSATSTELCYENLNRSCVRSPYSPGPRLILYAVFGFGAALAVCGNLLVMTSILHFRQLHSPANFLVVSLACADFLVGLTVMPFSTVRSVEGCWYFGESYCKLHTCFDVSFCYCSIFHLCFISVDRYIAVSDPLTYPTRFTAFVSGKCITFSWLLSTIYGFSLLYTGANEAGLEDLVSALTCVGGCQLAVNQSWVFINFLLFLIPTLVMITVYSKIFLIAKQQAQNIEKMSKQTARASDSYKDRVAKRERKAAKTLGIAVAAFLLSWLPYFIDSIIDAFLGFITPTYVYEILVWIAYYNSAMNPLIYAFFYPWFRKAIKLIVSGKVLRENSSTTNLFPE

SEQ ID NO: 18 - DNA, 1055 bp, Mus musculus
  mTAAR7c pseudogene
atgactacaggtgatgacagttttccttggaaccaagacagcatcctgagcagagatctgttctctgccacatctgcagagctgtgctatgagaacctgaacaggtcctgtgtcaggagcccatactcctcaggccctcgcctcatcctctatgcagtctttggctttggggctgtgctggctgtgtgtggaaacctcctggtgatgacatcaattcttcatttcaggcagctgcactctcctgccaacttcctggtggcatccctggcctgtgctgacttcttggtaggggtgatggtgatgccctttagcacagtgaggtctgtggagggctgctggtactttggggagatttactgtaaattccattcttgttttgaagggtcattctgttactcttctatcttccacttgtgtttcatctctattgatagatatattgcagtcagtgaccccctgatctaccccaccaggtttactgcatctgtttctggcaagtgcatcaccttctcctgtccaccatctacagcttttccctcctttatacaggggccaatgaagctgggctgaaggatctagtgagtgccctcacctgtgtgggtggctgtcaagtcccagtgaatcaaagctgggtcttcatcaacttcctattatttcttatccccacccttgtgatgataactgtctactctaagattttcctcattgctaaacagcaggctcagaacattgagaagatgagcaagcagactgccagggcatcagacagctacaaggacagggtggccaagagggagaggaaagcagccaaaacgctgggcattgcagtggctgccttcctcctttcatggctgccatacttcattgactcctttattgatgccttcctagggtatgtgtatgaaatcctagtttggatcgcttactataactcagccatgaaccctttgatttatgctttcttttatccttggtttcgaaaagccatcaaactcattgtcactggcaaaatcctgagagtgaattcctcaaccaccaacctgtttcccgaatag

SEQ ID NO: 19 - DNA, 1055 bp, Mus musculus; identical to SEQ ID NO: 18
  mTAAR7c fixed pseudogen

SEQ ID NO: 20 - protein, 351 aa, Mus musculus
  mTAAR7c fixed
MTTGDDSFPWNQDSILSRDLFSATSAELCYENLNRSCVRSPYSSGPRLILYAVFGFGAVLAVCGNLLVMTSILHFRQLHSPANFLVASLACADFLVGVMVMPFSTVRSVEGCWYFGEIYCKFHSCFEGSFCYSSIFHLCFISIDRYIAVSDPLIYPTRFTASVSGKCITFSLSTIYSFSLLYTGANEAGLKDLVSALTCVGGCQVPVNQSWVFINFLLFLIPTLVMITVYSKIFLIAKQQAQNIEKMSKQTARASDSYKDRVAKRERKAAKTLGIAVAAFLLSWLPYFIDSFIDAFLGYVYEILVWIAYYNSAMNPLIYAFFYPWFRKAIKLIVTGKILRVNSSTTNLFPE

SEQ ID NO: 21 - DNA, 1055 bp, Mus musculus; identical to SEQ ID NO: 18
  mTAAR7d

SEQ ID NO: 22 - protein, 358 aa, Mus musculus
  mTAAR7d
MATGDDSFPWDQDSILSRDLFSATSTELCYENLNRSCVRSPYSPGPRLILYAVFGFGAVLAVCGNLLVMTSILHFRQLHSPANFLVASLACADFLVGVMVMPFSMVRSVEGCWYFGESYCKFHSCFEGSFCYSSLFHLCFISVDRYIAVSDPLTYPTRFTASVSGKCITFSWLLSIIYSFSLLYTGANDAGLEDLVSALTCVGGCQIAVNQTWVFINFLLFLIPTLVMITVYSKIFLIAKQQAQNIEKMSKQTARASESYKDRVTKRERKAAKTLGIAVAAFLLSWLPYFIDSIIDAFLGFITPTYVYEILVWIVYYNSAMNPLIYAFFYSWFRKAIKLIVSGKILRENSSTTNLFPE

SEQ ID NO: 23 - DNA, 1077 bp, Mus musculus
  mTAAR7e
atggctacaggtgatgacagttttctctgggaccaagacagcatcctgagcagagatctgttctctgccacatctgcagagctgtgctatgagaacctgaacaggtcctgtgtcagaagcccatactccccaggccctcgcctcatcctctatgcagtctttggctttggggctgtgctggctgtgtgtggaaacctcctggtgatgacatcaattcttcatttcaggcagctgcactctcctgccaacttcctggtggcatccctggcctgtgctgacttcttggtgggtctgactgtgatgcccttcagcacagtgaggtctgtggagggctgctggtactttggggagatttactgtaaattacacacttgttttgatgtatctttctgtagttcttctattttccacttgtgcttcatctctgttgatagatatattgcagtcagtgaccccctgatctaccccaccaggtttactgcatctgtttctaacaagtgcatcactttctcctggcttctgtccatcagctatgggttttcccttatttacacaggagcaagtgaagctgggctagaggatctagtgagtgccctcacctgtgtgggtggctgtcaacttgcagtgaatcaaagctgggtctttatcaactttctattatttcttatccccacccttgtgatgataactgtctactctaagattttcctcattgctaaacagcaggctcagaacattgagaagatgagcaagcagactgccagggcatcagacagctacaaggacagggtggccaagagggagaggaaagcagccaaaaccctgggcatcgcagtggctgccttcctcctttcatggctgccatacttcattgactccttcattgatgccttcctggggttcatcacgcccacgtatgtgtatgaaatcctagtttggatcgcttactataactcagccatgaaccctttgatttatgctttcttttatccttggtttcgaaaagccatcaaactcactgttactggcaaaatcttgagggaaaattcctcaaccaccaacctgtttcctgagtag

SEQ ID NO: 24 - protein, 358 aa, Mus musculus
  mTAAR7e
MATGDDSFLWDQDSILSRDLFSATSAELCYENLNRSCVRSPYSPGPRLILYAVFGFGAVLAVCGNLLVMTSILHFRQLHSPANFLVASLACADFLVGLTVMPFSTVRSVEGCWYFGEIYCKLHTCFDVSFCSSSIFHLCFISVDRYIAVSDPLIYPTRFTASVSNKCITFSWLLSISYGFSLIYTGASEAGLEDLVSALTCVGGCQLAVNQSWVFINFLLFLIPTLVMITVYSKIFLIAKQQAQNIEKMSKQTARASDSYKDRVAKRERKAAKTLGIAVAAFLLSWLPYFIDSFIDAFLGFITPTYVYEILVWIAYYNSAMNPLIYAFFYPWFRKAIKLTVTGKILRENSSTTNLFPE

SEQ ID NO: 25 - DNA, 1077 bp, Mus musculus
  mTAAR7f
atgtctatagctgatgaaactgtttcctggaaccaagacagcatcctgagcagagatctgttctctgccacatctgcagagctgtgctatgagaacctgaacaggtcctgtgtcagaagcccatactccccaggccctcgcctcatcctctatgcagtctttggctttggggctgtgctggctgtgtgtggaaacctcctggtgatgacatcaattcttcatttcaggcagctgcactctcctgccaacttcctggtggcatccctggcctgtgctgacttcttggtaggggtgatggtgatgccctttagcatggtgaggtctgtggagggctgctggtactttggagacagttactgtaaattacacacttgttttgatgtatctttctgctattgttctctcttccacttgtgcttcatctctgttgatagatatattgcagtcagtgaccccctggcctaccccaccaggtttactgcatctgtttctggcaagtgcatcaccttctcctggctcctgtccatcagctatgggttttccctcatttacacaggagcaagtgaagctgggctggaggatctagtgagttccctcacctgtgtgggtggctgtcaaatcgcagtgaatcaaacctgggtctttatcaacttctcagtatttctcattcccacccttgtgatgataactgtctactctaagattttcctcattgctaaacagcaggctcagaacattgagaagatgagcaagcagactgccagggcatcagacagctacaaggacagggtggccaagagggagaggaaagcagccaaaaccctgggcatcgcagtggctgccttcctcctttcatggctgccatacttcattgactcctttattgatgccttcctagggttcatcacgcccacgtatgtgtatgaaatcctagtttggattgtttactataactcagccatgaaccctttgatttatgctttcttttatccttggtttcgaaaagccatcaaactcactgttactggcaaaatcttgagggaaaattcctcaaccaccaatctgttttctgagtag

SEQ ID NO: 26 - protein, 358 aa, Mus musculus
  mTAAR7f
MSIADETVSWNQDSILSRDLFSATSAELCYENLNRSCVRSPYSPGPRLILYAVFGFGAVLAVCGNLLVMTSILHFRQLHSPANFLVASLACADFLVGVMVMPFSMVRSVEGCWYFGDSYCKLHTCFDVSFCYCSLFHLCFISVDRYIAVSDPLAYPTRFTASVSGKCITFSWLLSISYGFSLIYTGASEAGLEDLVSSLTCVGGCQIAVNQTWVFINFSVFLIPTLVMITVYSKIFLIAKQQAQNIEKMSKQTARASDSYKDRVAKRERKAAKTLGIAVAAFLLSWLPYFIDSFIDAFLGFITPTYVYEILVWIVYYNSAMNPLIYAFFYPWFRKAIKLTVTGKILRENSSTTNLFSE

SEQ ID NO: 27 - DNA, 1035 bp, Mus musculus
  mTAAR8a
atgaccagcaacttttcccaaccagccctgcagctctgctatgagaacacgaatggatcctgtattaaaactccctattctccagggccccgggtcatcctgtacatggtctatggctttggggctgtgctggcagtgtgtgggaacctcctggtggtgatttcagttctccatttcaagcagctgcactctccagccaattttctcatcgcctctctggccagtgctgacttcttggtgggcatctctgtgatgcccttcagcatggtcaggtccattgagagctgctggtactttggagatgcattttgtagccttcacagttgttgtgatgtggctttttgttactcttctgtcctccacctctgcttcatctcagtggacaggtacatcgctgtcacggaccctctggtctatcccaccaagttcacagtgtctgtgtctggaatttgcatcagcatctcctggattctgcccctggtgtacagcagtgcagtgttttacacaggcatcagtgctaaggggattgaaagcttagtaagtgctctaaattgtgtagggggctgccaaattgttatcaatcaagactttgttttgataagttttcttctattcttcatacctacccttgttatgatcattctttacagcaaaatatttttggtagccaaacagcaagctgtaaaaattgaaacttctgtaagtggcaacagaggtgaatcatcctcagagagtcacaaagccagagtggccaagagagagaggaaggctgcaaaaaccttgggggtcactgtggtggcttttatggtctcgtggctcccatacacaattgacgcattggttgatgctttcatgggcttcatcactcctgcctatgtctatgaaatctgttgctggggtacctattataactcagccatgaaccctttgatttatgctttcttttttccttggtttaagaaagccataaagcttattttaagtggggaaattctaaagggacattcatcaactgcaaatttattttcagagtaa

SEQ ID NO: 28 - protein, 344 aa, Mus musculus
  mTAAR8a
MTSNFSQPALQLCYENTNGSCIKTPYSPGPRVILYMVYGFGAVLAVCGNLLVVISVLHFKQLHSPANFLIASLASADFLVGISVMPFSMVRSIESCWYFGDAFCSLHSCCDVAFCYSSVLHLCFISVDRYIAVTDPLVYPTKFTVSVSGICISISWILPLVYSSAVFYTGISAKGIESLVSALNCVGGCQIVINQDFVLISFLLFFIPTLVMIILYSKIFLVAKQQAVKIETSVSGNRGESSSESHKARVAKRERKAAKTLGVTVVAFMVSWLPYTIDALVDAFMGFITPAYVYEICCWGTYYNSAMNPLIYAFFFPWFKKAIKLILSGEILKGHSSTANLFSE

SEQ ID NO: 29 - DNA, 1035 bp, Mus musculus
  mTAAR8b
atgaccagcaacttttcccaaccagccctgcagctctgctatgagaacacgaatggatcctgtattaaaactccctattctccagggccccgggtcatcctgtacatggtctttggctttggggctgtgctggcagtgtgtgggaacctcctggtggtgatttcagttctccatttcaagcagctgcactctccagccaattttctcatcgcctctctggccagcgctgacttcttggtgggcatctctgtgatgcccttcagcatggtcaggtccattgagagctgctggtactttggagatgcattttgtagccttcacagttgctgtgatgtggctttttgttactcttctgccctccacctgtgcttcatctcagtggacaggtacattgctgtcacagaccctctggtctatcccaccaagttcacagtgtctgtgtctggaatttgcatcagcatctcctggattctgcccctggtatacagcagtgcagtgttctacacaggcatcagtgctaaggggattgaaagcttagtaagtgctctgaattgtgtagggggctgccaaatcgttgtcaatcaagactgggttttgatagattttcttctatttttcatacctacccttgttatgatcattctttacagcaaaatatttttggtagccaaacagcaagctgtaaaaattgaaacttctgtaagtgacaacagaggtgaatcatcctcagagagtcacaaagccagagtggccaagagagagaggaaggctgcaaaaaccttgggggtcactgtggtggcttttatggtctcgtggctcccatacacaattgactcattggttgatgctttcgtgggcttcattactcctgcctatgtctatgaaatctgttgctggagtgcctattataactcagccatgaaccctttgatttatgctttcttttatccttggtttaggaaagccataaagcttattttaagtggggagattctaaagagtcattcatctaccatgagtttgttttcagagtaa

SEQ ID NO: 30 - protein, 344 aa, Mus musculus
  mTAAR8b
MTSNFSQPALQLCYENTNGSCIKTPYSPGPRVILYMVFGFGAVLAVCGNLLVVISVLHFKQLHSPANFLIASLASADFLVGISVMPFSMVRSIESCWYFGDAFCSLHSCCDVAFCYSSALHLCFISVDRYIAVTDPLVYPTKFTVSVSGICISISWILPLVYSSAVFYTGISAKGIESLVSALNCVGGCQIVVNQDWVLIDFLLFFIPTLVMIILYSKIFLVAKQQAVKIETSVSDNRGESSSESHKARVAKRERKAAKTLGVTVVAFMVSWLPYTIDSLVDAFVGFITPAYVYEICCWSAYYNSAMNPLIYAFFYPWFRKAIKLILSGEILKSHSSTMSLFSE

SEQ ID NO: 31 - DNA, 1035 bp, Mus musculus
  mTAAR8c
atgaccagcaacttttcccaaccagccctgcagctctgctatgagaacacgaatggatcctgtattaaaactccctattctccagggccccgggtcatcctgtacatggtctatggctttggggctgtgctggcagtgtgtgggaacctcctggtggtgatttcagttctccatttcaagcagctgcactctccagccaattttctcatcgcctctctggccagtgctgacttcttggtgggcatctctgtgatgcccttcagcatggtcaggtccattgagagctgctggtactttggagatgcattttgtagccttcacagttgctgtgatgtggctttttgttactcttctgccctccacctgtgcttcatctcagtggacaggtacatagctgtcacggaccctctggtctatcccaccaagttcacagtgtctgtgtctggaatttgcatcagcatctcctggattctgcccctggtatacagcagtgcagtgttctacacaggcatcagtgctaaggggattgaaagcttagtaagtgctctgaattgtgtagggggctgccaagttgttgtcaatcaagactgggttttgataagttttcttttattcttcatacctaccgttgttatgatcattctttacagcaaaatatttttggtagccaaacagcaagctgtaaaaattgaaacttctgtaagtggcaacagaggtgaatcatcctcagagagtcacaaagccagagtggccaagagagagaggaaggctgcaaaaaccttgggggtcactgtggtggcttttatggtctcgtggctcccatacacaattgatgcattggttgatgctttcatgggcttcatcactcctgcctatgtctatgaaatctgttgctggagtgcctattataactcagccatgaaccctttgatttatgctttcttttatccttggtttaggaaagccataaagcttattttaagtgggaaaattctaaagggacattcatcaactacaaatttattttcagagtaa

SEQ ID NO: 32 - protein, 344 aa, Mus musculus
  mTAAR8c
MTSNFSQPALQLCYENTNGSCIKTPYSPGPRVILYMVYGFGAVLAVCGNLLVVISVLHFKQLHSPANFLIASLASADFLVGISVMPFSMVRSIESCWYFGDAFCSLHSCCDVAFCYSSALHLCFISVDRYIAVTDPLVYPTKFTVSVSGICISISWILPLVYSSAVFYTGISAKGIESLVSALNCVGGCQVVVNQDWVLISFLLFFIPTVVMIILYSKIFLVAKQQAVKIETSVSGNRGESSSESHKARVAKRERKAAKTLGVTVVAFMVSWLPYTIDALVDAFMGFITPAYVYEICCWSAYYNSAMNPLIYAFFYPWFRKAIKLILSGKILKGHSSTTNLFSE

SEQ ID NO: 33 - DNA, 1047 bp, Mus musculus
  mTAAR9
atgacaagcgacttctccccagaaccgcccatggagctctgctatgagaatgtgaacggatcctgcattaaaagctcttacgcaccctggcctcgagccatcctctatggggtcctcggtttgggagccctgctggcagtgtttgggaacttactggtcatcattgccatcctccactttaaacagctgcacacgcctacgaacttcctggtggcctccctggcctgtgctgacttcttggtgggggtgacagtgatgcccttcagcacggtgcgttctgtggagagctgctggtactttggggagagttactgtaagttccacacgtgtttcgacacctccttctgttttgcgtctctgtttcatttatgctgcatctccattgaccggtacattgcagttacagatccactgacctatccgaccaagttcactgtatcggtttctggactgtgcattgctctctcttggttcttttctgtcacatacagcttttccatcttttacacgggagccaatgaggaagggattgaggaattagtggttgctctgacctgtgtgggaggctgccaggctccactgaatcaaaattgggttttactttgtttccttttgttctttctgcccactgtggtcatggtgtttctatatggtcggatatttttggtggcaaagtatcaggctaggaagatagagggtacagccaaccaagctcaggcctcctctgagagctataaggaaagagtagccaaaagagagaggaaggctgccaagaccttggggatcgccatggctgcatttctcgtgtcctggctgccatacattattgatgcggttattgacgcttacatgaacttcataactcctgcgtacgtctatgagatattagtgtggtgtgtttactacaattcagctatgaaccccttgatatacgcctttttttatccttggtttcgtaaggcaataaaacttattgtgagtggcaaagtcttcagggctgattcatcaacaactaatctattctctgaagaggcaggtgcaggttaa

SEQ ID NO: 34 - protein, 348 aa, Mus musculus
  mTAAR9
MTSDFSPEPPMELCYENVNGSCIKSSYAPWPRAILYGVLGLGALLAVFGNLLVIIAILHFKQLHTPTNFLVASLACADFLVGVTVMPFSTVRSVESCWYFGESYCKFHTCFDTSFCFASLFHLCCISIDRYIAVTDPLTYPTKFTVSVSGLCIALSWFFSVTYSFSIFYTGANEEGIEELVVALTCVGGCQAPLNQNWVLLCFLLFFLPTVVMVFLYGRIFLVAKYQARKIEGTANQAQASSESYKERVAKRERKAAKTLGIAMAAFLVSWLPYIIDAVIDAYMNFITPAYVYEILVWCVYYNSAMNPLIYAFFYPWFRKAIKLIVSGKVFRADSSTTNLFSEEAGAG

SEQ ID NO: 35 - DNA, 18 bp, Mus musculus
  musTAR2_5_01, primer
atgaatacacccgacccc

SEQ ID NO: 36 - DNA, 19 bp, Mus musculus
  musTAR2_3_01, primer
ctaaggatgtgcaggatgc

SEQ ID NO: 37 - DNA, 18 bp, Mus musculus
  musGPR58_5_01, primer
atggcatcttttgaagcc

SEQ ID NO: 38 - DNA, 27 bp, Mus musculus
  musGPR58_3_01, primer
ctattctgtttctttttgagtaaacaa

SEQ ID NO: 39 - DNA, 25 bp, Mus musculus
  musGPR57_5_01, primer
atggatctaatatacattcccgaag

SEQ ID NO: 40 - DNA, 22 bp, Mus musculus
  musGPR57_3_01, primer
ttaatgtgcttcaggaaaaaga

SEQ ID NO: 41 - DNA, 18 bp, Mus musculus
  musTAR2_5_01, primer
atgaatacacccgacccc

SEQ ID NO: 42 - DNA, 19 bp, Mus musculus
  musTAR2_3_01, primer
ctaaggatgtgcaggatgc

SEQ ID NO: 43 - DNA, 19 bp, Mus musculus
  musPNR_5_01, primer
atgagagctgtcctcctcc

SEQ ID NO: 44 - DNA, 25 bp, Mus musculus
  musPNR_3_01, primer
tcagtcatggtataaatcaacagtc

SEQ ID NO: 45 - DNA, 20 bp, Mus musculus
  musTAR4_5_01, primer
atgggcagtaactcgtctcc

SEQ ID NO: 46 - DNA, 25 bp, Mus musculus
  musTAR4_3_01, primer
ttatatttgctcagagaacaagttg

SEQ ID NO: 47 - DNA, 19 bp, Mus musculus
  musTAR8_5_01, primer
atggacaaattggttgacc

SEQ ID NO: 48 - DNA, 22 bp, Mus musculus
  musTAR8_3_01, primer
ctactcaggaaacaagttggtg

SEQ ID NO: 49 - DNA, 24 bp, Mus musculus
  musTAR12_5_01, primer
tggctacagataatgacagttttc

SEQ ID NO: 50 - DNA, 22 bp, Mus musculus
  musTAR12_3_01, primer
ctactcaggaaacaagttggtg

SEQ ID NO: 51 - DNA, 25 bp, Mus musculus
  musTAR17_5_01, primer
atgactacaggtgatgacagttttc

SEQ ID NO: 52 - DNA, 20 bp, Mus musculus
  musTAR17_3_01, primer
ctattcgggaaacaggttgg

SEQ ID NO: 53 - DNA, 20 bp, Mus musculus
  musTAR14_5_01, primer
atggctacaggtgatgacag

SEQ ID NO: 54 - DNA, 20 bp, Mus musculus
  musTAR14_3_01, primer
ctattcaggaaacaggttgg

SEQ ID NO: 55 - DNA, 20 bp, Mus musculus
  musTAR9_5_01, primer
atggctacaggtgatgacag

SEQ ID NO: 56 - DNA, 20 bp, Mus musculus
  musTAR9_3_01, primer
ctattcaggaaacaggttgg

SEQ ID NO: 57 - DNA, 26 bp, Mus musculus
  musTAR6_5_01, primer
atgtctatagctgatgaaactgtttc

SEQ ID NO: 58 - DNA, 23 bp, Mus musculus
  musTAR6_3_01, primer
ctactcagaaaacagattggtgg

SEQ ID NO: 59 - DNA, 19 bp, Mus musculus
  musTAR11_5_01, primer
atgaccagcaacttttccc

SEQ ID NO: 60 - DNA, 26 bp, Mus musculus
  musTAR11_3_01, primer
ttactctgaaaataaatttgcagttg

SEQ ID NO: 61 - DNA, 19 bp, Mus musculus
  musTAR7_5_01, primer
atgaccagcaacttttccc

SEQ ID NO: 62 - DNA, 26 bp, Mus musculus
  musTAR7_3_01, primer
ttactctgaaaacaaactcatggtag

SEQ ID NO: 63 - DNA, 19 bp, Mus musculus
  musTAR10_5_01, primer
atgaccagcaacttttccc

SEQ ID NO: 64 - DNA, 28 bp, Mus musculus
  musTAR10_3_01, primer
ttactctgaaaataaatttgtagttgat

SEQ ID NO: 65 - DNA, 19 bp, Mus musculus
  musTAR3_5_01, primer
atgacaagcgacttctccc

SEQ ID NO: 66 - DNA, 18 bp, Mus musculus
  musTAR3_3_01, primer
ttaacctgcacctgcctc

SEQ ID NO: 67 - protein, 339 aa, Homo sapiens
MMPFCHNIINISCVKNNWSNDVRASLYSLMVLIILTTLVGNLIVIVSISHFKQLHTPTNWLIHSMATVDFLLGCLVMPYSMVRSAEHCWYFGEVFCKIHTSTDIMLSSASIFHLSFISIDRYYAVCDPLRYKAKMNILVICVMIFISWSVPAVFAFGMIFLELNFKGAEEIYYKHVHCRGGCSVFFSKISGVLTFMTSFYIPGSIMLCVYYRIYLIAKEQARLISDANQKLQIGLEMKNGISQSKERKAVKTLGIVMGVFLICWCPFFICTVMDPFLHYIIPPTLNDVLIWFGYLNSTFNPMVYAFFYPWFRKALKMMLFGKIFQKDSSRCKLFLELSS

SEQ ID NO: 68 - protein, 351 aa, Homo sapiens
MAVSSEQHELSHFKRTQTKKEKFNCSEYGNRSCPENERSLGVRVAMYSFMAGSIFITIFGNLAMIISISYFKQLHTPTNFLILSMAITDFLLGFTIMPYSMIRSVENCWYFGLTFCKIYYSFDLMLSITSIFHLCSVAIDRFYAICYPLLYSTKITIPVIKRLLLLCWSVPGAFAFGVVFSEAYADGIEGYDILVACSSSCPVMFNKLWGTTLFMAGFFTPGSMMVGIYGKIFAVSRKHAHAINNLRENQNNQVKKDKKAAKTLGIVIGVFLLCWFPCFFTILLDPFLNFSTPVVLFDALTWFGYFNSTCNPLIYGFFYPWFRRALKYILLGKIFSSCFHNTILCMQKESE

SEQ ID NO: 69 - protein, 337 aa, Homo sapiens
MRAVFIQGAEEHPAAFCYQVNGSCPRTVHTLGIQLVIYLACAAGMLIIVLGNVFVAFAVSYFKALHTPTNFLLLSLALADMFLGLLVLPLSTIRSVESCWFFGDFLCRLHTYLDTLFCLTSIFHLCFISIDRHCAICDPLLYPSKFTVRVALRYILAGWGVPAAYTSLFLYTDVVETRLSQWLEEMPCVGSCQLLLNKFWGWLNFPLFFVPCLIMISLYVKIFVVATRQAQQITTLSKSLAGAAKHERKAAKTLGIAVGIYLLCWLPFTIDTMVDSLLHFITPPLVFDIFIWFAYFNSACNPIIYVFSYQWFRKALKLTLSQKVFSPQTRTVDLYQE

SEQ ID NO: 70 - protein, 345 aa, Homo sapiens
MSSNSSLLVAVQLCYANVNGSCVKIPFSPGSRVILYIVFGFGAVLAVFGNLLVMISILHFKQLHSPTNFLVASLACADFLVGVTVMPFSMVRTVESCWYFGRSFCTFHTCCDVAFCYSSLFHLCFISIDRYIAVTDPLVYPTKFTVSVSGICISVSWILPLMYSGAVFYTGVYDDGLEELSDALNCIGGCQTVVNQNWVLTDFLSFFIPTFIMIILYGNIFLVARRQAKKIENTGSKTESSSESYKARVARRERKAAKTLGVTVVAFMISWLPYSIDSLIDAFMGFITPACIYEICCWCAYYNSAMNPLIYALFYPWFRKAIKVIVTGQVLKNSSATMNLFSEHI

SEQ ID NO: 71 - protein, 342 aa, Homo sapiens
MTSNFSQPVVQLCYEDVNGSCIETPYSPGSRVILYTAFSFGSLLAVFGNLLVMTSVLHFKQLHSPTNFLIASLACADFLVGVTVMLFSMVRTVESCWYFGAKFCTLHSCCDVAFCYSSVLHLCFICIDRYIVVTDPLVYATKFTVSVSGICISVSWILPLTYSGAVFYTGVNDDGLEELVSALNCVGGCQIIVSQGWVLIDFLLFFIPTLVMIILYSKIFLIAKQQAIKIETTSSKVESSSESYKIRVAKRERKAAKTLGVTVLAFVISWLPYTVDILIDAFMGFLTPAYIYEICCWSAYYNSAMNPLIYALFYPWFRKAIKLILSGDVLKASSSTISLFLE

SEQ ID NO: 72 - protein, 348 aa, Homo sapiens
MVNNFSQAEAVELCYKNVNESCIKTPYSPGPRSILYAVLGFGAVLAAFGNLLVMIAILHFKQLHTPTNFLIASLACADFLVGVTVMPFSTVRSVESCWYFGDSYCKFHTCFDTSFCFASLFHLCCISVDRYIAVTDPLTYPTKFTVSVSGICIVLSWFFSVTYSFSIFYTGANEEGIEELVVALTCVGGCQAPLNQNWVLLCFLLFFIPNVAMVFIYSKIFLVAKHQARKIESTASQAQSSSESYKERVAKRERKAAKTLGIAMAAFLVSWLPYLVDAVIDAYMNFITPPYVYEILVWCVYYNSAMNPLIYAFFYQWFGKAIKLIVSGKVLRTDSSTTNLFSEEVETD

SEQ ID NO: 73 - protein, 335 aa, Rattus norvegicus
MHLCHNSANISHTNSNWSRDVRARATSLYSLISLIILTTLVGNLIVIISISHFKQLHTPTNWLLHSMAVVDFLLGCLVMPYSMVRTVEHCWYFGELFCKLHTSTDIMLSSASILHLAFISIDRYYAVCDPLRYKAKINLAAIFVMILISWSLPAVFAFGMIFLELNLEGVEEQYHNQVFCLRGCFPFFSKVSGVLAFMTSFYIPGSVMLFVYYRIYFIAKGQARSINRANLQVGLEGESRAPQSKETKAAKTLGIMVGVFLLCWCPFFFCMVLDPFLGYVIPPTLNDTLNWFGYLNSAFNPMVYAFFYPWFRRALKMVLFGKIFQKDSSRSKLFL

SEQ ID NO: 74 - protein, 339 aa, Rattus norvegicus
MTSFEAQQETFDCSEYGNGSCPENERSLGVRAAMYSLMAGAIFITIFGNLVMIISISYFKQLHTPTNLLILSMAVTDFLLGFTIMPYSMVRSVENCWYFGLTFCKIHYSFDLMLSITSIFHLCSVAIDRFYAICHPLHYCTKMTIPVVKRLLLVCWSVPGAFAFGVVFSEAYADGIEGYDILVACSSSCPVMFNKLWGTTLFVAGFFTPSSMMVGIYGKIFAVSKKHARVIDNLPENQNNQMRKDKKAAKTLGIVMGVFLLCWFPCFFTILLDPFLNFSTPAILFDALTWFGYFNSTCNPLIYGFFYPWFRRALRYILLGKIFSSHFHNTNLFTQKETE

SEQ ID NO: 75 - protein, 342 aa, Rattus norvegicus
MDLIYIPEDLSSCPKFGNKSCPPTNRSFRVRLIMYLLMTGAMVITIFGNLVIIISISHFKQLHSPTNFLILSMATTDFLLGFVIMPYSMVRSVESCWYFGDSFCKFHASFDMMLSLTSIFHLCSIAIDRFYAVCAPLHYTTTMTASMIKRLLFFCWAAPALFSFGLVLSEANVSGMQSYEILIACFNFCALTFNKFWGTILFTTCFFTPGSIMVGIYGKIFIVSRRHARALGNMPENTKGAGRNLSKKKDRKAAKTLGIVMGVFLACWLPCFLAVLIDPYLDYSTPIIVLDLLVWLGYFNSTCNPLIHGFFYPWFRKALEHIVSGKIFRSNSDTANLFPEAH

SEQ ID NO: 76 - protein, 347 aa, Rattus norvegicus
MNSPDLWYSPETQFCFAAANNSCPRKARPALVVCAMYLVMIGAIVMTMLGNMVVIISIAHFKQLHSPTNFLILSMATTDFLLSCVVMPFSMVRSIESCWYFGDLFCKVHSCCDIMLCTTSIFHLCFISVDRHYAVCDPLHYVTQITVGVVGVFLLISWSVPILFAFGLVFSELNLIGAEDFVAAIDCTGLCVLIFNKLWGVLASFIAFFLPGAIMVGIYIHIFTVARKHARKIGPGPRTKRALSESKMKATSGKESKATKTLSIVMGVFVLCWLPFFVLTITDPFIGFTTPEDLYNVFLWLGYFNSTFNPIIYGMFYPWFRKALRMIVTGTIFRSDSSTSSLHPAHP

SEQ ID NO: 77 - protein, 344 aa, Rattus norvegicus
MRAVLLPGSGEQPAAFCYQVNGSCPRTVHPLAIRVLIYLACAVGMLITVLGNLFVVFAVSYFKVLHTPTNFLLLSLALADMLLGLLVLPLSTVRSVESCWFFGDFLCRLHTYLDTLFCLTSIFHLCFISIDRHCAICDPLLYPSKFTVRIALRYIAAGWGIPAAYTAFFLYTDVVERALSQWLEEMPCVGSCQLLFNKFWGWLNFPAFFIPCLIMISLYLKIFVVATRQAQQIRTLSQSLSGAVKRERKAAKTLGIAVGIYLVCWLPFTVDTLRATTAARVDSLLNFVTPPLVFDIFIWFAYFNSACNPIIYVFSYRWFRKALKLLLSREILSPRTQTADLFHD

SEQ ID NO: 78 - protein, 345 aa, Rattus norvegicus
MGSNSSPPAVLQLCYENVNGSCVKTPYSPGPRVLLYAVFGFGAVLAVFGNLLVMISILHFKQLHSPTNFLIASLACADFWVGVSVMPFSMVRSIESCWYFGRSFCTFHTCCDVAFCYSSLFHLSFISIDRYIAVTDPLVYPTKFTVSVSGICISISWILPLAYSGAVFYTGVYADGLEEVSDAVNCVGGCQVVVNQNWVLIDFLSFLIPTLVMIILYGNIFLVARQQAKKIETVGNKAESSSESYKARVARRERKAAKTLGITVVAFMISWLPYSIDSLVDAFMGFITPAYIYEICVWCAYYNSAMNPLIYALFYPWFKKAIKVIMSGQVFKNSSATMNLFSEQI

SEQ ID NO: 79 - protein, 344 aa, Rattus norvegicus
MTSNFSQAPLQLCYENVNASCIKTPYSPGLRVLLYMVFGFGAVLAVCGNLLVVISVLHFKQLHSPANFLIASLASADFLVGISVMPFSMVRSIESCWYFGDTFCSLHSCCDAAFCYSSLFHLCFISVDRYIAVTDPLVYPTKFTVSVSGICISISWILPLVYSSAVFYTGISATGIENLVSALNCVGGCQIVVNQDWVLIDFLLFLIPTLVMIILYSKIFLVAKQQAVKIETSISGSKGESSLESHKARVAKRERKAAKTLGVTVVAFMVSWLPYTIDTLIDAFMGFITPAYVYEICCWSAYYNSAMNPLIYAFFYPWFRKAIKLILSGEILKSHSSTMSLFSE

SEQ ID NO: 80 - protein, 344 aa, Rattus norvegicus
MTSNFSQATLQLCYENVNASCIKTPYSPGLRVLLYMVFGFGAVLAVCGNLLVVISVLHFKQLHSPANFLIASLASADFLVGISVMPFSMVRSIESCWYFGDTFCSLHSCCDAAFCYSSLFHLCFISVDRYIAVTEPLVYPTKFTMSVSGICISISWILPLVYSSAVFYTGISATGIENLVSALNCVGGCQVAINQDWVLISFLLFFIPTLVMIILYSKIFLVAKQQAVKIETSISGSKGESSLESHKARVAKRERKAAKTLGVTVMAFMVSWLPYTIDTLIDAFMGFITPAYVYEICGWIAYYNSAMNPLIYAFFYPWFRKAIKLILSGKILKGHSSTTSLFSE

SEQ ID NO: 81 - protein, 344 aa, Rattus norvegicus
MTSNFSQATLQLCYENVNASCIKTPYSPGLRVLLYMVFGFGTVLAVCGNLLVVISVLHFKQLHSPANFLIASLASADFLVGISVMPFSMVRSIESCWYFGDTFCSLHSCCDVAFCYSSALHLCFISVDRYIAVTDPLVYPTKFTVSVSGICISISWILPLVYSSAVFYTGISAMGIENLVSALNCVGGCQVVVNQDWVLISFLLFFIPTLVMIILYSKIFLVAKQQAVKIETSVSGSKGESSLESHKARVAKRERKAAKTLGVTVLAFIVSWLPYTIDTLIDAFMGFITPAYVYEFCCWSAYYNSAMNPLIYAFFYPWFRKAMKLILSGKILKGHSSTTSLFSE

SEQ ID NO: 82 - protein, 338 aa, Rattus norvegicus
MELCYENVNGSCIKSSYSPWPRAILYAVLGLGALLAVFGNLLVITAILHFKQLHTPTNFLVASLACADFLVGVTVMPFSTVRSVEGCWYFGDTYCKFHTCFDTSFCFASLFHLCCISIDRYVAVTDPLTYPTKFTISVSGVCIALSWFFSVTYSFSIFYTGANEEGIEELVVALTCVGGCQAPLNQNWVLLCFLLFFLPTVVMVFLYGRIFLVAKQQARKIEGSANQPQASSESYKERVARRERKAAKTLGIAMAAFLVSWLPYIIDAVIDAYMNFITPAYVYEILVWCVYYNSAMNPLIYAFFYPWFRKAIKLIVSGKVFRADSSRTNLFSEEAGAG

SEQ ID NO: 83 - protein, 358 aa, Rattus norvegicus
MDKLVDNFLSGQSRTMSEDLLSASSPQLCYENLNGSCIRSPYSPGPRLILYAVFGFGAVLAVCGNLLVMTSILHFRQLHSPANFLVASLACADFLVGLTVMPFSTVRSVEGCWYFGDTYCKFHSCFEGSFCYSSIFHLCFISVDRYIAVSDPLIYPTRFTASVSGKCITFSWLLSIIYSFSLLYTGANEAGLEDLVSALTCVGGCQIAVNQSWVFINFLLFLVPTLVMMTVYSKIFLIAKQQAQNIEKMSKQTTRASESYKDRVAKRERKAAKTLGIAVAAFLLSWLPYFIDSIIDAFLGFITPTYVYEILVWIAYYNSAMNPLIYAFFYPWFRKAIKLIVTGKILRQNSSVTNLFPE

SEQ ID NO: 84 - protein, 358 aa, Rattus norvegicus
MATDDDRFPWDQDSILSRDLLSASSMQLCYEKLNRSCVRSPYSPGPRLILYAVFGFGAVLAVCGNLLVMTSILHFRQLHSPANFLVASLACADFLVGLTVMPFSMVRSVEGCWYFGDIYCKFHSSFDGSFCYSSIFHLCFISADRYIAVSDPLIYPTRFTASVSGKCITFSWLLSIIYSFSLFYTGVNEAGLEDLVSALTCVGGCQIAVNQSWVFINFLLFLVPALVMMTVYSKIFLIAKQQAQNIEKMGKQTARASESYKDRVAKRERKAAKTLGIAVAAFLLSWLPYFIDSIIDAFLGFVTPTYVYEILVWIGYYNSAMNPLIYAFFYPWFRKAIKLIVTGKILRENSSATNLFPE

SEQ ID NO: 85 - protein, 358 aa, Rattus norvegicus
MATDDASFPWDQDSILSRDLLSASSSQLCYENLNRSCIRSPYSPGPRLILHAVFGFSAVLAVCGNLLVMTSILHFRQLHSPANFLVASLACADLLVGLTVMPFSMVRSVEGCWYFGDIYCKFHSSFDVSFCYCSLFHLCFISVDRYIAVSDPLIYPTRFTASVSGKCITFSWFLSIIYSFSLLYTGASEAGLEDLVSALTCVGGCQLAVNQSWVFINFLLFLVPTLVMMTVYSKVFLIAKQQAQNIEKIGKQTARASESYKDRVAKRERKAAKTLGITVAAFLLSWLPYFIDSIIDAFLGFITPTYVYEILVWIAYYNSAMNPLIYAFFYPWFRKAIKLIVTGKILRENSSATNLFPE

SEQ ID NO: 86 - protein, 358 aa, Rattus norvegicus
MATDDDSFPWDQDSILSRDLLSASSLQLCYENLNRSCVRSPYSPGSRLILYAVFGFGAVLAVCGNLLVMTSILHFRQLHSPANFLVASLACADLLVGLTVMPFSMVRSVEGCWYFGNTYCKFHSCFEGSFCYSSLFHLCFISLDRYIAVSDPLIYPTRFTASISGKCITFSWLLSIIYSFSLLYTGANEAGLEDLVSALTCVGGCQVAVNQSWVFINFLLFLVPALVMMTVYSKIFLIAKQQAQNIEKMSKQTARASESYKDRVAKRERKAAKTLGIAVAAFLLSWLPYFIDSIIDAFLGFITPTYMYEILVWIVYYNSAMNPLIYAFFYPWFRKAIKLIVTGKILRENSSTINLFPE

SEQ ID NO: 87 - protein, 358 aa, Rattus norvegicus
MRVDDDRFPWDQDSILSRDLLSASSLQLCYENLNRSCVRSPYSPGPRLILYAVFGFGAVLAVCGNLLVMTSILHFRQLHSPANFLVASLACADLLVGLTVMPFSMVRSVEGCWYFGDTYCKLHTSFDVSFCYCSLFHLCFISVDRYIAVSDPLIYPTRFTASVSGKCITFSWLLSIIYGFPLIYTGASEAGLEDLVSALTCVGGCQIPMNQKFVLINFLLFLVPTLVMMTVYSKIFLIARQQAQNIEKMRKQTARASESYKDRVCKRERKAAKTLGIAVAAFLLSWLPYFIDSIIDAFLGFITPTYVYEILIWIVYYNSSMNPLIYAFFYPWFRKATKLIVTGKILRENSSTINLFPE

SEQ ID NO: 88 - protein, 358 aa, Rattus norvegicus
MATDDGSFPWTQESILSRDLLSALSPQLCYENLNRSCVRSPYSPGSRLILYAVFGFGAVLAVCGNLLVMTSILHFRQLHSPANFLVASLACADLLVGLTVMPFSMVRSVEGCWYFGDSYCKLHSCFDISFCSSSLLHLCFISVDRHIAVSDPLIYPTRFTASVSGKYITFSWLLSIIYGFSLIYTGASEAGLEDLVSALTCVGGCQVAVNQSWVFINFLLFLVPALVMMTVYSKIFLIAKQQAQNMEKMSKQTARASDSYKDRVAKRERKAAKTLGIAVAAFLLSWLPYFVDSIIDAFLGFITPTYVYEILAWIAYYNSAMNPLIYAFFYPWFRKAIKLIVTGKILKENSSTINLFPE

SEQ ID NO: 89 - protein, 358 aa, Rattus norvegicus
MATDDESFPWDQDSILSRDLLSALSPQLCYENLNRSCVRSPYSPGPRLILYAVFGFGAVLAVCGNLLVMTSILHFRQLHSPANFLVASLACADLLVGLTVMPFSMVRSVEGCWYFGDSYCKLHTSFDMSFCCSSLLHLCFISVDRYIAVSDPLIYPIRFTASVSGKCITFSWFLSIIYGFSLIYTGASEAGLKDLVSALSCVGGCQIPMNQSCVLINFLLFLVPTLVMMTVYSKIFLIAKQQAQNMEKMSKQTTRASDSYKDRVAKRERKAAKTLGIAVAAFLLSWLPYLIDSIIDAFLGFITPSYVYEILVWIVYYNSAMNPLIYAFFYPWFRNAIKLIVTGKILKQNSSTTNLFSE

SEQ ID NO: 90 - DNA, 1032 bp, Homo sapiens
  CDS (1)..(1029)
atggatctaacttatattcccgaagacctatccagttgtccaaaatttgtaaataaatcctgtcctcccaccaaccgctcttttcatgtccaggtgataatgtattcggttatgactggagccatgattatcccactattcggaaacttggttataatggtttccatatcgcatttcaaacagcttcactctcccacaaactttctgatcctctccatggcaaccacggactttctgctgggttttgtcattatgccatacagcataatgcgatcagtggagagttgctggtactttggggatggcttttgtaaattccacacaagctttgacatgatgctcagactgacctccattttccacctctgttccattgctattgaccgattttatgccgtgtgttaccctttacattacacaaccaaaatgacgaactccaccataaagcaactgctggcattttgctggtcagttcctgctcttttttcttttggtttagttctatctgaggccgatgtttccggtatgcagagctataagatacttgttgcttgcttcaatttctgtgcccttactttcaacaaattctgggggacaatattgttcactacatgtttctttacccctggctccatcatggttggtatttatggcaaaatctttatcgtttccaaacagcatgctcgagtcatcagccatgtgcctgaaaacacaaagggggcagtgaaaaaacacctatccaagaaaaaggacaggaaagcagcgaagacactgggtatagtaatgggggtgtttctggcttgctggttgccttgttttcttgctgttctgattgacccatacctagactactccactcccatactaatattggatcttttagtgtggctccggtacttcaactctacttgcaaccctcttattcatggcttttttaatccatggtttcagaaagcattcaagtacatagtgtcaggaaaaatatttagctcccattcagaaactgcaaatttgtttcctgaagcacattaa

SEQ ID NO: 91 - protein, 343 aa, Homo sapiens
MDLTYIPEDLSSCPKFVNKSCPPTNRSFHVQVIMYSVMTGAMIIPLFGNLVIMVSISHFKQLHSPTNFLILSMATTDFLLGFVIMPYSIMRSVESCWYFGDGFCKFHTSFDMMLRLTSIFHLCSIAIDRFYAVCYPLHYTTKMTNSTIKQLLAFCWSVPALFSFGLVLSEADVSGMQSYKILVACFNFCALTFNKFWGTILFTTCFFTPGSIMVGIYGKIFIVSKQHARVISHVPENTKGAVKKHLSKKKDRKAAKTLGIVMGVFLACWLPCFLAVLIDPYLDYSTPILILDLLVWLRYFNSTCNPLIHGFFNPWFQKAFKYIVSGKIFSSHSETANLFPEAH

SEQ ID NO: 92 - protein, 35 aa, Homo sapiens
LMMSHTDILSSIFTFSFGFWFFTLIWGYVGGCSVF

SEQ ID NO: 93 - protein, 35 aa, Mus musculus
LMMSHTDILSSIYAFSFGFWFFTLYWGYVGGCSPF

SEQ ID NO: 94 - protein, 35 aa, Rattus norvegicus
LIMSHTDILSSIFAFSFGFWFFMLNWGYVRGCFPF

SEQ ID NO: 95 - protein, 35 aa, Homo sapiens
VMMSHSDILCTIFASAFGFWFFTFLWGYIGLCVLF

SEQ ID NO: 96 - protein, 35 aa, Rattus norvegicus; identical to SEQ ID NO: 95

SEQ ID NO: 97 - protein, 35 aa, Mus musculus
IMMSHSDILCTIFASAFGFWFFTFLWGYIGLCVLF

SEQ ID NO: 98 - protein, 35 aa, Homo sapiens
FMMSYYDLLSIIFLFGFGFWCFILTWGYVSSCPVF

SEQ ID NO: 99 - protein, 35 aa, Mus musculus
LMMSHYDLLSIIFLFGFSFWCFILTWGYVSSCPVF

SEQ ID NO: 100 - protein, 35 aa, Rattus norvegicus; identical to SEQ ID NO: 99

SEQ ID NO: 101 - protein, 35 aa, Homo sapiens
VMMSHTDMLRLIFLFCFGFWCFVLVWRYVNFCALF

SEQ ID NO: 102 - protein, 35 aa, Mus musculus
FMMSHADMLSLIFLFCFGFWCFVLVWGYVNFCALF

SEQ ID NO: 103 - protein, 35 aa, Rattus norvegicus
LMMSHADMLSLIFLFCFGFWCFVLVWGYINFCALF

SEQ ID NO: 104 - protein, 35 aa, Homo sapiens
ACLSHTDTFCLIYLNLFCYWFTTFIWAYMGSCQLL

SEQ ID NO: 105 - protein, 35 aa, Mus musculus
ACLSHTDTFCLIYLNAFCYWFTTFIWAYMGSCQLF

SEQ ID NO: 106 - protein, 35 aa, Rattus norvegicus; identical to SEQ ID NO: 105

SEQ ID NO: 107 - protein, 35 aa, Rattus norvegicus
VFMSHTDMFCCLYINLFTFWYLSLVWVYIQSCVLN

SEQ ID NO: 108 - protein, 35 aa, Rattus norvegicus
VFMSHTDIFCFLYINLFTFWYLSLVWVYIQNCVLN

SEQ ID NO: 109 - protein, 35 aa, Homo sapiens
VFMSHTDVFCYLYTDSFTFWYSSCCWAYLGGCQTV

SEQ ID NO: 110 - protein, 35 aa, Mus musculus
VFMSHTDVFCYLYIDSFTFWYSSCVWAYLGGCQVV

SEQ ID NO: 111 - protein, 35 aa, Rattus norvegicus
VFMSHTDVFCYLYIDSFTFWYSSCVWAYVGGCQVV

SEQ ID NO: 112 - protein, 35 aa, Mus musculus
VYMSHSDVFCYVYISLFTFWYTACCWTYLGGCQII

SEQ ID NO: 113 - protein, 35 aa, Mus musculus
VYMSHSDVFCYAYISLFTFWYTACCWAYLGGCQVV

SEQ ID NO: 114 - protein, 35 aa, Homo sapiens
AFMSHSDVFCYVYIDLFTFWYTICCWAYLGGCQIV

SEQ ID NO: 115 - protein, 35 aa, Mus musculus
VFMSHSDVFCYAYIDLFTFWYTSCCWAYLGGCQIV

SEQ ID NO: 116 - protein, 35 aa, Rattus norvegicus
VFMSHSDAFCYLYIDLFTFWYTTCCWAYLGGCQIV

SEQ ID NO: 117 - protein, 35 aa, Rattus norvegicus
VFMSHSDVFCYAYISLFTFWYTTCCWAYLGGCQVV

SEQ ID NO: 118 - protein, 35 aa, Rattus norvegicus
VFMSHSDAFCYLYISLFTFWYTTCGWAYLGGCQVI

SEQ ID NO: 119 - protein, 35 aa, Homo sapiens
VLMSHTDTFCFLYLCLFNFWYLALVWVYLGGCQAL

SEQ ID NO: 120 - protein, 35 aa, Mus musculus
VLMSHTDTFCFLYLCLFTFWYIALVWVYLGGCQAL

SEQ ID NO: 121 - protein, 35 aa, Rattus norvegicus; identical to SEQ ID NO: 120

SEQ ID NO: 122 - protein, 35 aa, Rattus norvegicus
VFMSHSDIFCSLYINLFAFWYFSLAWAYLGGCQVV

SEQ ID NO: 123 - protein, 35 aa, Mus musculus
VFMSHTDVFCSIYINLFTFWYFSLVWAYLGGCQLV

SEQ ID NO: 124 - protein, 35 aa, Rattus norvegicus
VFMSHSDVFCYLYINLFTFWYFSLVWAYLGGCQLV

SEQ ID NO: 125 - protein, 35 aa, Mus musculus
VFMSHTDVFCYIYINLFTFWYFSLVWAYLGGCQLV

SEQ ID NO: 126 - protein, 35 aa, Mus musculus
VFMSHTDVFCYLYINVFTFWYFSLVWVYLGGCQIV

SEQ ID NO: 127 - protein, 35 aa, Rattus norvegicus
VFMSHTDVFCYLYINLFTFWYFSLIWVYLGGCQIM

SEQ ID NO: 128 - protein, 35 aa, Rattus norvegicus
VFMSHSEGFCYIYINLFTFWYFSLVWAYLGGCQIV

SEQ ID NO: 129 - protein, 35 aa, Mus musculus; identical to SEQ ID NO: 128

SEQ ID NO: 130 - protein, 35 aa, Mus musculus
VFMSHSEGFCYIYINLFTFWYFSLVWAYLGGCQVV

SEQ ID NO: 131 - protein, 35 aa, Mus musculus
VFMSHSEGFCYLYINLFTFWYFSLVWVYLGGCQIV

SEQ ID NO: 132 - protein, 35 aa, Rattus norvegicus
VFMSHSEGFCYLYINLFAFWYFSLVWVYLGGCQVV

SEQ ID NO: 133 - protein, 35 aa, Rattus norvegicus
VFMSHSDGFCYIYINLFAFWYFSLVWGYLGGCQIV

SEQ ID NO: 134 - protein, 35 aa, Rattus norvegicus
VFMSHTDMFRCLYINFFAFWYLSLTWVYLGGCQIV

SEQ ID NO: 135 - protein, 16 aa, Homo sapiens
  MOD_RES (3)..(4): Variable amino acid
NSXXNPXXYXXXYXWF

SEQ ID NO: 136 - protein, 16 aa, Rattus norvegicus
  MOD_RES (3)..(4): Variable amino acid
NSXXNPXXXXXXYXWF